(19) United States
(12) Patent Application Publication
(10) Pub. No.: US 2025/0177401 A1
(43) Pub. Date: Jun. 5, 2025

(54) COMBINATION THERAPY OF SOLID CANCER

(71) Applicant: MOR RESEARCH APPLICATIONS LTD., Ramat Gan (IL)

(72) Inventors: Salomon STEMMER, Givat Shmuel (IL); Neta MOSKOVITS, Bet Gamliel (IL)

(21) Appl. No.: 18/975,802

(22) Filed: Dec. 10, 2024

Related U.S. Application Data
(63) Continuation of application No. 17/417,625, filed on Jun. 23, 2021.

(30) Foreign Application Priority Data
Dec. 23, 2018 (IL) 263905

(51) Int. Cl. A61K 31/519; A61K 31/404; A61K 31/44; A61K 31/506; A61K 45/06; A61P 35/04

(52) U.S. Cl. CPC A61K 31/519; A61K 31/404; A61K 31/44; A61K 31/506; A61K 45/06; A61P 35/04

(57) ABSTRACT

A method for treating adenoid cystic carcinoma (ACC) is provided, comprising co-administering at least inhibitor of cyclin-dependent kinase 4/6 (CDK 4/6) and at least one multi-targeted receptor tyrosine kinase inhibitor (mtRTKI). The CDK 4/6 inhibitor may be at least one of palbociclib, abemaciclib and ribociclib and the mtRTKI may be at least one of sunitinib, sorafenib and pazopanib. A combination therapy with palbociclib and sunitinib provides a synergic effect in treatment of ACC.

This application is a continuation-in-part (CIP) of Ser. No. 17/417,625 filed Jun. 23, 2021, which is a 371 of PCT/IL2019/051400 filed Dec. 23, 2019, which claims benefit of Israeli Patent Application No. 263905 filed Dec. 23, 2018. The entire contents of each of the above applications are hereby incorporated herein by reference for all purposes.

### FIELD OF THE INVENTION

The present disclosure relates to combinational therapies of solid cancer comprising co-administration of inhibitors of cyclin-dependent kinases 4 and 6 and inhibitors of receptor tyrosine kinases, particularly, but not exclusively, combination therapy comprising palbociclib and sunitinib for treatment of adenoid cystic carcinoma.

### BACKGROUND OF THE INVENTION

One of the key hallmarks of cancer is deregulation of the cell-cycle, resulting in aberrant cell proliferation. In normal cells, a tight regulation of the cell cycle via regulatory proteins keeps the division cycles in control. Cyclin-dependent kinases 4 and 6 (CDK4/6) play a central role in this important regulation, and their inhibitors trigger cell cycle arrest. The clinical development of the CDK4/6 inhibitors has changed clinical practice in the setting of endocrine-receptor positive breast cancer. Results of pivotal phase II and III trials investigating these CDK4/6 inhibitors in patients with endocrine receptor-positive advanced breast cancer have demonstrated a significant improvement in progression-free survival, with a safe toxicity profile. These agents were approved for use in combination with aromatase inhibitor as a first and second line of treatment of hormone positive HER2 negative metastatic breast cancer.

Most solid tumors show deregulation of multiple signaling pathways. An important mechanism in signal transduction pathways in cells is protein phosphorylation, which is carried out by protein kinases, including receptor tyrosine kinases (RTKs). These RTKs regulate the fundamental processes of proliferation, differentiation, migration, metabolism and anti-apoptotic signaling of the cell, as well as interaction with the microenvironment to regulate angiogenesis. It is recognized now that multi targeted therapy with tyrosine kinase inhibitors which targets several signaling pathways simultaneously, is more effective than single targeted therapy. One example of a multi-targeted receptor tyrosine kinase (RTK) inhibitor is sunitinib, a small molecule that was approved by the FDA for the treatment of renal cell carcinoma (RCC) and imatinib-resistant gastrointestinal stromal tumor.

The current standard of care treatments for cancer are defined and contain mostly chemotherapeutic agents, biologic agents, targeted therapy and hormones (in hormone positive tumors). For patients who fail the first lines (1-2 lines) of treatment, no strict guidelines are defined for further treatment in most malignancies. Combinational treatment of cancer has been recently adopted by physicians (Si-Yong Qin et al., 2018, Biomaterials, 171:178-197). Examples of such combinations are combination of chemotherapy drugs, combination of chemotherapy plus gene therapy, and chemotherapy plus immunotherapy. Bollard et al. (Gut, 2017, 66:1286-1296) discussed encouraging results in preclinical models of treating hepatocellular carcinoma by palbociclib alone or in combination with sorafenib, showing an additive effect. Small et al., (Oncotarget, 2017, 8 (56):95116-95134) showed positive results of combining abemaciclib with sunitinib in renal cell carcinoma xenograft tumors. Uras et al., (Blood, 2016, 127 (23):2890-2902) reported treatment of patients having acute myeloid leukemia harboring FLT3-IDT mutation with FLT3 inhibitors in combination with palbociclib.

Although there are some general considerations that may be contemplated in order to enable a successful combinational therapy, yet, actually, there are no clear rules predicting which combination therapy will work. According to Chou (Pharmacol. Rev., 2006, 58:621-681), synergism or antagonism needs to be determined but cannot be predicted a priori. In each case, a careful examination must be performed.

### SUMMARY OF THE INVENTION

There is a long existing need for development of additional safe and proven combinational therapies to treat cancer.

The present disclosure relates to combinations of two distinct types of anticancer drugs for achieving synergistic therapeutic effects. Specific combinations of inhibitors of cyclin-dependent kinases 4 and 6 (CDK 4/6) together with a multi-targeted receptor tyrosine kinase inhibitor (mtRTKI) have been found by the present inventors to control various types of solid tumors more efficiently than the current standard of care and provide a very efficient control of tumor growth. For example, administration of a combination of palbociclib and sunitinib to mice implanted with different types of malignancies prevented the growth of these malignancies, and furthermore, in some cases reduced the initial size of the tumor. In addition, the combination treatment completely prevented the development of tumors in several different human cancers assessed in immune deficient mice models. In fact, such combination treatment provided a synergistic anti-cancer effect in a plurality of cancer models. Interestingly, results of treatment with this drug combination as well as with other combinations of CDK 4/6 inhibitors and mtRTKIs were completely unexpected since some combinations provided advantageous results while other combinations were additive or less effective.

It is difficult, if not impossible, to predict the effect that a combination of drugs would have. The three main effects that may be observed when two or more compounds are administered together are: additive effect—the most common; a synergistic effect—the effect of a combination is more than a simple summation of individual effects; and an antagonism—the effect of a combination is less than a simple summation of individual effects.

It is shown in the present disclosure that combination of palbociclib and sunitinib provided a synergistic effect in a plurality of cancer models. The synergy was shown in several types of carcinomas, neuroendocrine tumors, Ewing sarcoma and in carcinosarcoma originating from different organs such as lung cancer, stomach cancer, breast cancer, ovarian cancer, colon cancer, pancreas cancer and cholangiocarcinoma. The successful treatment of patients afflicted with adenoid cystic carcinoma (ACC) is demonstrated.

Interestingly, synergism was not seen in hepatitis C positive hepatocellular carcinoma, which showed weak to moderate additive effect. In appendix cancer model, concomitant administration of the two drugs provided effect which was worse than the effect of sunitinib alone. Combinations of other CDK 4/6 inhibitors and mtRTKIs such as a combination of ribociclib with pazopanib, and combination of abemaciclib with sorafenib provided additive effects without showing synergy in some of the tested models. Combinations of palbociclib and sorafenib showed synergistic effect, e.g., in ovarian and lung cancer models. Attaining an additive effect of two anticancer drugs is an achievement by itself, which is not easy to obtain and may significantly improve the quality of treatment (e.g., by reducing the dose or obtaining better effect). Thus, obtaining a synergic effect is highly unpredictable and it is impossible to anticipate a priori which of the combinations will provide such an effect.

According to one aspect, the present disclosure relates to a combination of an inhibitor of cyclin-dependent kinase 4/6 (CDK 4/6) and a multi-targeted receptor tyrosine kinase inhibitor (mtRTKI) for use in treating cancer, provided that the cancer is not hepatocellular carcinoma or renal cell carcinoma. According to some embodiments, the CDK 4/6 inhibitor is selected from palbociclib, abemaciclib and ribociclib and the mtRTKI is selected from sunitinib, sorafenib and pazopanib. In some embodiments, a disclosed combination of a CDK 4/6 inhibitor and an mtRTKI is useful in treating solid cancer, for example, wherein the CDK 4/6 is palbociclib and the mtRTKI is selected from sunitinib and sorafenib. In some embodiments, a disclosed combination of drugs comprises palbociclib and sunitinib for use in treating a solid cancer, wherein the cancer is not hepatocellular carcinoma. In some embodiments, a disclosed combination comprises of palbociclib and sorafenib for use in treating a solid cancer, provided that the cancer is not hepatocellular carcinoma.

Any of the combinations disclosed herein may be used in treatment of one or more of the following cancers: a carcinoma, a neuroendocrine tumor, carcinosarcoma, Ewing sarcoma, sarcoma, lymphoma, and melanoma. In some embodiments, the carcinoma is adenoid cystic carcinoma (ACC). According to some embodiments, the cancer is selected from lung cancer, stomach cancer, breast cancer, ovarian cancer, neuroendocrine cancer, pancreas cancer, cholangiocarcinoma, bone cancer, bile ducts cancer, small bowel cancer, liposarcoma, brain tumor, uterus cancer, cervical cancer, head and neck cancer, parotid cancer, salivary gland cancer, gallbladder cancer, bile duct cancer, adrenal cancer and colon cancer.

When a combination of palbociclib and sunitinib is for use in treating solid cancer, palbociclib may be administered in a dose of from 20 to 250 mg/day and sunitinib and administered in a dose of from 10 to 125 mg/day.

When a combination of palbociclib and sorafenib is for use in treating solid cancer, palbociclib may be administered in a dose of from 20 to 250 mg/day and sorafenib may be administered in a dose of from 200 to 800 mg/day.

According to some embodiments, the daily administered dose of at least one of the compounds in a combination is lower than the standard daily dose of this compound when administered alone. In some embodiments, a disclosed combination is a synergistic combination comprising palbociclib and sunitinib for use in treating solid cancer, provided that the cancer is not hepatocellular carcinoma or appendix cancer. In some embodiments, a disclosed combination is a synergistic combination comprising palbociclib and sorafenib, for use in treating solid cancer, provided that the cancer is not hepatocellular carcinoma.

In another aspect, the present disclosure relates to a pharmaceutical composition comprising an inhibitor of CDK 4/6 and a multi-targeted receptor tyrosine kinase inhibitor (mtRTKI), wherein the CDK 4/6 inhibitor is selected from palbociclib, abemaciclib and ribociclib, and the mtRTKI is selected from sunitinib, sorafenib and pazopanib. In some embodiments, the pharmaceutical composition comprises palbociclib and sunitinib in doses of, e.g., from 20 to 150 mg/day palbociclib and from 5 to 75 mg/day of sunitinib. In some embodiments, the pharmaceutical composition comprises palbociclib and sorafenib in doses of, e.g., from 20 to 150 mg/day palbociclib and from 50 to 800 mg sorafenib.

In some embodiments, a disclosed pharmaceutical composition comprises ribociclib and pazopanib in amounts ranging, e.g., from 50 to 300 mg ribociclib and from 100 to 500 mg of pazopanib.

In some embodiments, a disclosed pharmaceutical composition comprises abemaciclib and sorafenib in amounts ranging, e.g., from 20 to 300 mg abemaciclib and from 50 to 300 mg of sorafenib.

Pharmaceutical compositions disclosed herein are useful in treating any of the solid cancers disclosed herein. In some embodiments, a contemplated pharmaceutical composition comprises palbociclib and sunitinib and is applied in the treatment of adenoid cystic carcinoma (ACC).

According to some embodiments, the pharmaceutical composition provides a synergic anticancer effect.

According to another aspect, the present disclosure relates to a method of treating a solid cancer in a subject in need thereof, the method comprises co-administering to the subject an inhibitor of cyclin-dependent kinase 4/6 (CDK 4/6) selected from palbociclib, abemaciclib and ribociclib, and a multi-targeted receptor tyrosine kinase inhibitor (mtRTKI) selected from sunitinib, sorafenib and pazopanib. In some embodiments, the cancer is not hepatocellular carcinoma or a renal cell carcinoma. In some embodiments, the method comprises administering palbociclib and sunitinib and provides a synergic anticancer effect. In some embodiments, the method comprises administering palbociclib and sorafenib and provides a synergic anticancer effect.

According to a further aspect, the present disclosure relates to a kit comprising at least one inhibitor of cyclin-dependent kinase 4/6 (CDK 4/6) and at least one multi-targeted receptor tyrosine kinase inhibitor (mtRTKI), and instructions for use, optionally wherein the CDK 4/6 inhibitor is palbociclib and the mtRTKI is selected from sunitinib and sorafenib.

According to some embodiments, the CDK 4/6 inhibitor and the mtRTKI are present as a pharmaceutical composition.

### BRIEF DESCRIPTION OF DRAWINGS

FIG. 1 shows the effect on tumor volume of a combo treatment comprising palbociclib and sunitinib in a colon cancer model (adenocarcinoma comprising KRAS mutation G12V);

FIG. 2 shows the effect on tumor volume of various doses of palbociclib and sunitinib in combo treatment in a colon cancer model;

FIG. 3 shows the effect on tumor volume of a combo treatment comprising palbociclib and sunitinib in a colon cancer model (well differentiated adenocarcinoma);

FIG. 4 shows the effect on tumor volume of various doses of palbociclib and sunitinib in combo treatment in a colon cancer model;

FIG. 5 shows the effect on tumor volume of a combo treatment comprising palbociclib and sunitinib in a colon cancer model (metastatic adenocarcinoma with wild-type RAS);

FIG. 6A shows the effect on tumor volume of a combo treatment comprising palbociclib and sunitinib in a stomach cancer model (poorly differentiated stomach adenocarcinoma (HER2 negative)); FIG. 6B shows the effect on tumor volume of standard treatment with a combination of oxaliplatin+5FU (SOC) on the same stomach cancer tissue;

FIG. 7 shows the effects on tumor volume of combo treatments comprising ribociclib and pazopanib or abemaciclib and sorafenib, and monotherapy with each of these drugs alone, in a stomach cancer model (poorly differentiated stomach adenocarcinoma (HER2 negative);

FIG. 8 shows the effects on tumor volume of combo treatments comprising palbociclib and sunitinib or ribociclib and pazopanib, and monotherapy with each of these drugs alone, in a breast cancer model (invasive ductal carcinoma (ER positive);

FIG. 9 shows the effects on tumor volume of a combo treatment comprising palbociclib and sunitinib in 5-FU resistant a breast cancer model;

FIG. 10 shows the effects on tumor volume of combo treatments comprising palbociclib and sunitinib) or ribociclib and pazopanib, and monotherapies with each of these drugs alone, in a triple negative breast cancer (TNBC) model. Treatment with cisplatin served as positive control;

FIG. 11 shows the effects on tumor volume of combo treatment comprising palbociclib and sunitinib, and monotherapies with each of these drugs alone, in a triple negative breast cancer (TNBC) model;

FIG. 12 shows the effects on tumor volume of a combo treatment comprising palbociclib and sunitinib, and monotherapies with each of these drugs alone, in a breast cancer model;.

FIG. 13 shows the dose effect on tumor size of combo treatment with palbociclib and sunitinib administered in various doses in a breast cancer model;

FIG. 14 shows the effects on tumor volume of combo treatments comprising palbociclib and sunitinib or palbociclib and sorafenib, and monotherapies with each of these drugs alone, a in a lung cancer model (poorly differentiated carcinoma).

FIGS. 15A and 15B show the effect on tumor volume of a combo treatment comprising palbociclib and sunitinib, monotherapies with each of these drugs alone, and SOC treatment with gemcitabine and carboplatin, in a lung cancer model (adenocarcinoma; KRAS mutation G12V). FIG. 15A shows progression of tumor with time of the different treatment groups. FIG. 15B shows tumors excised at the last day of the experiment (day 60) from mice of the different treatment groups (M represent one mouse);

FIG. 16 shows the effect on tumor volume of a combo treatment comprising palbociclib and sunitinib, monotherapies with each of these drugs alone, and SOC with carboplatin, in a metastatic neuroendocrine ovarian cancer model;

FIG. 17 shows the effects on tumor volume of combo treatments comprising palbociclib and sunitinib or palbociclib and sorafenib, and monotherapies with each of these drugs alone, in an ovarian cancer (metastatic high grade serous carcinoma with BRCA1 mutation) model;

FIG. 18 shows the effects on tumor volume of a combo treatment comprising palbociclib and sunitinib, monotherapies with each of these drugs alone and SOC treatment with doxorubicin, in a carcinosarcoma model;

FIG. 19 shows the effects on tumor volume of a combo treatment comprising palbociclib and sunitinib, monotherapies with each of these drugs alone and SOC treatment with gemcitabine and cisplatin ( [symbol] ) in a pancreatic cancer (adenocarcinoma tumor);

FIG. 20 shows the effects on tumor volume of various doses of palbociclib and sunitinib administered in combination in a pancreatic cancer model;

FIG. 21 shows the effect on tumor volume of a combo treatment with palbociclib and sunitinib, monotherapies with each of these drugs alone, and SOC treatment with gemcitabine and cisplatin, in a cholangiocarcinoma model;

FIG. 22 shows the effects on tumor volume of various of doses of palbociclib and sunitinib administered in combination in a cholangiocarcinoma cancer model;

FIG. 23 shows the effect on tumor volume of a combo treatment comprising palbociclib and sunitinib, and monotherapies with each of these drugs alone and with sorafenib, in a hepatocellular carcinoma model;

FIG. 24 shows the effects on tumor volume of a combo treatments comprising palbociclib and sunitinib or palbociclib and sorafenib, and monotherapies with palbociclib and sunitinib (each alone), in a model of appendix carcinoma;

FIG. 25 shows the effect on tumor volume of a combo treatment comprising ribociclib and pazopanib, and monotherapy with each of these drugs alone in, a model of appendix carcinoma;

FIG. 26 shows the effect on tumor volume of a combo treatment comprising palbociclib and sunitinib, monotherapies with each of these drugs alone and SOC treatment with folfox+avastin, in a model of Ewing sarcoma;

FIG. 27 are photographs of a patient diagnosed with high-grade sinonasal adenoid cystic carcinoma malignancy with squamous differentiation in the left maxillary sinus. The photographs were taken before combo treatment and 3 weeks post combo treatment with palbociclib and sunitinib with increased dose of sunitinib; and

FIG. 28 are photographs of a patient diagnosed with adenoid cystic carcinoma T3N0M0 (the tumor has extended into the surrounding anatomical structures but did not invade critically nearby structures (T3), there was no lymph node involvement (N0), and no signs of distant metastasis (M0)). The photographs were taken before combo treatment and 8 weeks post combo treatment with palbociclib and sunitinib.

### DETAILED DESCRIPTION OF THE INVENTION

The present disclosure provides efficient combination therapy, also referred to herein as “combo treatments” or simply “combo”, of a wide range of cancer malignancies, based on a combination of at least one inhibitor of cyclin-dependent kinase 4/6 and at least one inhibitor of receptor tyrosine kinase.

According to one aspect, the present disclosure relates to a combination of an inhibitor of cyclin-dependent kinase 4/6 (CDK 4/6) and a multi-targeted receptor tyrosine kinase inhibitor (mtRTKI) for use in treating solid cancer, wherein, in some embodiments, the cancer is not hepatocellular carcinoma or a renal cell carcinoma. The CDK 4/6 inhibitor may be at least one of palbociclib, abemaciclib or ribociclib. The mtTKI may be at least one of sunitinib, sorafenib or pazopanib. In some embodiments, a disclosed combination of CDK 4/6 inhibitor and mtTKI is used for treating adenoid cystic carcinoma (ACC).

The terms “cyclin-dependent kinase 4”, “cell division protein kinase 4” and “CDK4” are used herein interchangeably and refer to a human enzyme (International Union of Biochemistry and Molecular Biology (IUBMB) enzyme classification (EC) number 2.7.11.22) encoded by the CDK4 gene. The terms “cyclin-dependent kinase 6”, and “CDK6” are used herein interchangeably and refer to human enzyme (EC number 2.7.11.22) encoded by CDK6 gene. The terms “inhibitor of cyclin-dependent kinase 4/6”, “inhibitor of CDK 4/6” and “CDK 4/6 inhibitor” are used herein interchangeably and refer to an inhibitor that inhibits the activity of both CDK4 and CDK6 enzymes.

The terms “receptor tyrosine kinase” and “RTK” are used herein interchangeably and refer to a human receptor tyrosine kinase having EC number 2.7.10.1. The term “multi-targeted receptor tyrosine kinase inhibitor” and “mtRTKI” are used herein interchangeably and refer to inhibitors that inhibit two or more receptor tyrosine kinases.

The CDK 4/6 inhibitor is optionally selected from palbociclib, abemaciclib or ribociclib and the mtRTKI is optionally selected from sunitinib, sorafenib or pazopanib.

In some embodiments, the combination is at least one of: a combination of palbociclib and sunitinib; a combination of palbociclib and sorafenib; a combination of palbociclib and pazopanib; a combination of abemaciclib and sorafenib; a combination of abemaciclib and pazopanib; a combination of abemaciclib and sunitinib; a combination of ribociclib and sunitinib; a combination of ribociclib and sorafenib; and/or a combination of ribociclib and pazopanib.

The terms “treating” or “treatment of” a condition, disorder or disease of a patient refers to taking steps to obtain beneficial or desired results with respect to the condition, disorder or disease of the patient, including clinical results. Beneficial or desired clinical results include, but are not limited to, ameliorating, abrogating, substantially inhibiting, slowing or reversing the progression of a disease, condition or disorder, substantially ameliorating or alleviating clinical or esthetical symptoms of a condition, disorder or disease, substantially preventing the appearance of clinical or esthetical symptoms of a disease, condition, or disorder, and protecting from harmful or annoying symptoms. Treating further refers to accomplishing one or more of the following: (a) reducing the severity of a disorder, condition or disease; (b) limiting development of symptoms characteristic of the disorder, condition or disease being treated; (c) limiting worsening of symptoms characteristic of a disorder, condition or disease being treated; (d) limiting recurrence of a disorder, condition or disease in patients that have previously had the disorder, condition or disease; and/or (e) limiting recurrence of symptoms in patients that were previously asymptomatic for the disorder, condition or disease. The term “treating” with respect to cancer should be understood to encompass, e.g., a treatment resulting in a decrease in tumor size; a decrease in rate of tumor growth; stasis of tumor size; a decrease in the number of metastasis; a decrease in the number of additional metastasis; a decrease in invasiveness of the cancer; a decrease in the rate of progression of the tumor from one stage to the next; inhibition of tumor growth in a tissue of a mammal having a malignant cancer; control of establishment of metastases; inhibition of tumor metastases formation; regression of established tumors as well as decrease in the angiogenesis induced by the cancer; inhibition of growth and proliferation of cancer cells and so forth. The term “treating cancer” as used herein should also be understood to encompass prophylaxis such as prevention as cancer may reoccur after previous treatment (including surgical removal), and prevention of cancer in an individual prone (genetically, due to lifestyle, chronic inflammation and the like) to develop cancer. As used herein, “prevention of cancer” is thus to be understood to include prevention of metastases, for example, after surgical procedures or after chemotherapy.

As used herein, the term “cancer” refers to all types of cancer, neoplasm or malignant tumors found in mammals. As used herein, the term “solid cancer” refers to a mass-type cancer formed in an organ, unlike blood cancer. The term solid cancer includes lymphomas, melanomas, neuroendocrine tumors, carcinomas, carcinosarcoma, and sarcomas. “Sarcomas” are cancers of the connective tissue, cartilage, bone, muscle, and so on. “Carcinomas” are cancers of epithelial (lining) cells. “Adenocarcinoma” refers to carcinoma derived from cells of glandular origin. Neuroendocrine tumors are derived from cells that release hormones into the blood. The terms “cancer” and “tumor” are used interchangeably throughout the present disclosure. Exemplary cancers that may be treated with a contemplated combo treatment include lymphoma, sarcoma, bladder cancer, bone cancer (e.g., Ewing sarcoma), brain tumor, cervical cancer, colon cancer, esophageal cancer, gastric cancer, head and neck cancer, kidney cancer, thyroid cancer (e.g., medullary thyroid cancer, medullary thyroid carcinoma, papillary thyroid cancer), prostate cancer, breast cancer (e.g., triple negative, ER positive, ER negative, chemotherapy resistant, Herceptin resistant, HER2 positive, doxorubicin resistant, tamoxifen resistant, ductal carcinoma including palbociclib resistant ductal carcinoma, lobular carcinoma, primary, metastatic), ovarian cancer, pancreatic cancer, lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, squamous cell carcinoma (e.g., head, neck, or esophagus), colorectal cancer, B cell lymphoma. Additional examples include cancer of the endocrine or exocrine system, cervix, liver, stomach, or uterus, mesothelioma, medulloblastoma, neuroblastoma, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, genitourinary tract cancer, malignant hypercalcemia, endometrial cancer, adrenal cortical cancer, neoplasms of the cancer of the pancreas (e.g., cancer of the pancreatic stellate cells), Paget's disease of the nipple, phyllodes tumors, lobular carcinoma, cancer of the hepatic stellate cells, or adenoid cystic carcinoma (ACC).

In some embodiments, the drug combination of the present disclosure is for use in treating solid cancer, wherein the solid cancer is selected from carcinoma, a neuroendocrine tumor, Ewing sarcoma, carcinosarcoma, lymphoma, melanoma, and sarcoma. In some embodiments, the solid cancer is selected from lung cancer, stomach cancer, small bowel cancer, breast cancer, ovarian cancer, neuroendocrine cancer, pancreas cancer, cholangiocarcinoma, gallbladder cancer, bile duct cancer, colon cancer, bone cancer, liposarcoma, brain tumor, uterus cancer, cervical cancer, head and neck cancer, parotid cancer, salivary gland cancer, and adrenal cancer. In some embodiments, the solid cancer is a metastasis originating from lung cancer, stomach cancer, small bowel cancer, breast cancer, ovary cancer, endocrine system cancer, cancer of the pancreas, gallbladder cancer, bile duct cancer, colon cancer, bone cancer, fat cells cancer, brain cancer, uterus cancer, cancer of the cervix, head and neck cancer, parotid cancer, salivary gland cancer, or adrenal gland cancer.

In some embodiments, solid cancer treated with a combination of CDK 4/6 inhibitor and mtRTKI is carcinoma. The treatable carcinoma is selected from medullary thyroid carcinoma (MTC), familial medullary thyroid carcinoma (FMTC), acinar carcinoma of the pancreas, adenoid cystic carcinoma, adenocarcinoma, carcinoma of adrenal cortex, adenocarcinoma in situ (AIS), basal cell carcinoma, basaloid squamous cells carcinoma (BSCC), basosquamous cell carcinoma (BCC), bronchiolar carcinoma, bronchogenic carcinoma, cholangiocellular carcinoma, chorionic carcinoma, colloid carcinoma, comedo carcinoma, corpus carcinoma, carcinoma en cuirasse, cylindrical carcinoma, cylindrical cell carcinoma, ductal carcinoma (including ductal carcinoma in situ (DCIS) and invasive ductal carcinoma (IDC)), embryonal carcinoma, epiermoid carcinoma, carcinoma epitheliale adenoides, exophytic carcinoma,, gelatiniforni carcinoma, gelatinous carcinoma, non-small cell lung carcinoma (NSCLC) with large or giant cells, glandular carcinoma, granulosa cell carcinoma, hair-matrix carcinoma, hematoid carcinoma, Hurthle cell carcinoma, hyaline carcinoma, hypernephroid carcinoma, infantile embryonal carcinoma, carcinoma in situ, intraepidermal carcinoma, lenticular carcinoma, lipomatous carcinoma, lobular carcinoma, lymphoepithelial carcinoma, medullary carcinoma, melanotic carcinoma, carcinoma molle, arcinoma muciparum, carcinoma mucocellulare, mucoepidermoid carcinoma, carcinoma mucosum, mucous carcinoma, carcinoma myxomatodes, nasopharyngeal carcinoma, small-cell lung carcinoma (SCLC), osteoid carcinoma, papillary carcinoma, periportal carcinoma, prickle cell carcinoma, pultaceous carcinoma, reserve cell carcinoma, schneiderian carcinoma, scirrhous carcinoma, signet-ring cell carcinoma, carcinoma simplex, solanoid carcinoma, spindle cell carcinoma, squamous cell carcinoma, carcinoma telangiectodes, transitional cell carcinoma, tubular carcinoma, or tuberous carcinoma.

Embodiments of the present disclosure pertain to combo treatment of various and any types of carcinomas, wherein the CDK 4/6 inhibitor in the drug combination is at least one of palbociclib, abemaciclib and ribociclib, and the mtRTKI in the drug combination is at least one of sunitinib, sorafenib and pazopanib. For example, in some embodiments, the combination is palbociclib and sunitinib, whereas in some embodiments the combination is palbociclib and sorafenib.

In some embodiments, a combination of the present disclosure is for use in treating an adenocarcinoma.

In some embodiments, a combination of the present disclosure is for use in treating adenoid cystic carcinoma (ACC).

Adenoid cystic carcinoma is a rare type of cancer that primarily arises in the salivary glands but can also develop in other areas such as the breast, trachea, lacrimal glands (around the eyes), and other parts of the respiratory tract. It is known for its slow growth but highly invasive nature, which makes it difficult to treat. ACC typically occurs during adulthood, around the ages of 40 to 60, but has also been diagnosed in children and adolescents.

Key Characteristics of ACC include:
(i) Growth pattern. ACC grows slowly but tends to invade surrounding tissues, including nerves, which can make it particularly painful and harder to remove surgically.
(ii) Histology. under a microscope, ACC has a distinct appearance, with cells that form patterns resembling small ducts or cystic spaces, often described as “cribriform” or “Swiss cheese-like”. These structures are typically filled with a mucous-like material or contain abnormal fibrous membranes.
(iii) Metastasis. ACC has a high tendency to spread (metastasize) to distant parts of the body, particularly the lungs, even years after the primary tumor has been removed. Despite its slow growth, it can be persistent and recurrent.
(iv) Symptoms. symptoms depend on the tumor's location. For example, in the salivary glands there is a painless mass or swelling, often around the mouth or jaw. Involvement of nerves can lead to pain or facial paralysis. Tumors in the respiratory tract may cause breathing difficulties.
(v) Treatment. (1) surgery: the primary treatment is surgical removal of the tumor, but because ACC tends to infiltrate surrounding tissues, complete excision is often difficult; (2) radiation therapy: often used post-surgery to reduce the risk of recurrence; and/or (3) chemotherapy: ACC tumors usually are resistant to chemotherapy with only a small proportion deriving significant or sustained benefits. Systemic therapy including chemotherapy has not been approved by the U.S. Food and Drug Administration for the treatment of ACC. However, patients with ACC who have progressive disease may be eligible to participate in a clinical trial of a specific drug therapy.
(vi) Prognosis. ACC is often associated with a good short-term prognosis due to its slow-growing nature, but the long-term outlook can be less favorable due to its tendency for local recurrence and delayed metastasis. Long-term monitoring is essential for patients.

In some embodiments, a disclosed combination of one or more CDK4/6 inhibitors and one or more mtRTKIs (for brevity referred to herein “combination”) is for use in treating neuroendocrine tumor. In some embodiments, a disclosed combination is for use in treating sarcoma. In some embodiments, the combination is for use in treating carcinosarcoma. In some embodiments, the combination is for use in treating Ewing sarcoma. In some embodiments, the combination is for use in treating lung cancer. In some embodiments, the combination is for use in treating stomach cancer. In some embodiments, the combination is for use in treating colon cancer. In some embodiments, the combination is for use in treating breast cancer. In some embodiments, the combination is for use in treating ovarian cancer. In some embodiments, the combination is for use in treating neuroendocrine cancer. In some embodiments, the combination is for use in treating pancreas cancer. In some embodiments, the combination is for use in treating cholangiocellular carcinoma (also termed cholangiocarcinoma). In some embodiments, the combination is for use in treating bone cancer. In some embodiments, the combination is for use in treating gallbladder or bile duct cancer.

According to any one of the embodiments disclosed herein, palbociclib may be administered in a dose of from 25 to 250, from 30 to 225, from 35 to 200, from 40 to 175, from 50 to 150, from 55 to 130, from 60 to 125, from 60 to 85 or from 75 to 100 mg/day.

According to any one of the embodiments disclosed herein, the sunitinib may be administered in a dose of from 10 to 125, from 15 to 110, from 20 to 100, from 25 to 75, from 20 to 40, from 30 to 70, from 35 to 60 or from 40 to 55 mg/day.

According to any one of the embodiments disclosed herein, ribociclib may be administered in a dose of from 50 to 800, from 60 to 700, from 70 to 600, from 80 to 500, from 100 to 400 or from 200 to 300 mg/day.

According to any one of the embodiments disclosed herein, pazopanib may be administered in a dose of from 100 to 950, from 150 to 900, from 200 to 850, from 300 to 800, from 400 to 700, or from 500 to 600 mg/day.

According to any one of the embodiments disclosed herein, abemaciclib may be administered in a dose of from 100 to 800, from 150 to 700, from 200 to 600, or from 300 to 500 mg/day.

According to any one of the embodiments disclosed herein, sorafenib may be administered in a dose of from 100 to 950, from 150 to 900, from 200 to 850, from 300 to 800, from 400 to 700, or from 500 to 600 mg/day.

In some embodiments, a disclosed combination comprises palbociclib and sunitinib for use in treating a solid cancer as defined herein. In accordance with these embodiments, palbociclib may be administered in a dose of from 20 to 300 mg/day, for example, a dose of from 25 to 150, from 50 to 125 or from 75 to 100 mg/day. Further according to these embodiments, sunitinib may be administered in a dose of from 5 to 150 mg/day, for example, a dose of from 5 to 50 mg/day, from 10 to 40 mg/day or from 20 to 30 mg/day.

In some embodiments, palbociclib is administered in a dose of from 25 to 250 mg/day and sunitinib is administered in a dose of from 10 to 125 mg/day. In some embodiments, palbociclib is administered in a dose of from 25 to 150 mg/day and sunitinib is administered in a dose of from 5 to 50 mg/day.

In some embodiments, palbociclib is administered in a dose of 75 mg/day in combination with 25 mg/day of sunitinib, and then the dose of sunitinib increases, for example, to 37.5 mg/day.

In some embodiments, the solid cancer treated with a combination of palbociclib and sunitinib as defined herein is adenoid cystic carcinoma (ACC). In some embodiments, the solid cancer treated with the combination is 5FU resistant or palbociclib resistant breast cancer.

In some embodiments, a disclosed combination comprises palbociclib and sorafenib for use in treating solid cancer. In accordance with these embodiments, palbociclib may be administered in a dose of from 20 to 300 mg/day, for example, dose of from 25 to 150, from 50 to 125 or from 75 to 100 mg/day. Further according to these embodiments, sorafenib may be administered in a dose of 100 to 950, for example, a dose of from 250 to 800, from 100 to 900, from 150 to 850, from 200 to 800, or from 300 to 700 mg/day.

In some embodiments, palbociclib is administered in a dose of from 25 to 150 mg/day and sorafenib is administered in a dose of from 50 to 1200 mg/day.

In some embodiments, the solid cancer treated with a combination of palbociclib and sorafenib as defined herein is at least one of lymphoma, melanoma, neuroendocrine tumor, carcinoma, carcinosarcoma or sarcoma. In some embodiments, the carcinoma is adenocarcinoma. In some embodiments, the treated cancer is Ewing sarcoma. Other solid cancers treatable with a combination of palbociclib and sorafenib include, but are not limited to, lung cancer, stomach cancer, colon cancer, breast cancer, ovarian cancer, neuroendocrine cancer, pancreas cancer, bone cancer, cholangiocellular carcinoma. liposarcoma, brain tumor, uterus cancer, cervical cancer, head and neck cancer, parotid cancer, salivary gland cancer, adrenal cancer and small bowel cancer. In some embodiments, the breast cancer is 5FU palbociclib resistant breast cancer.

In some embodiments, a disclosed combination comprises ribociclib and pazopanib for use in treating solid cancer. In accordance with these embodiments, ribociclib may be administered in a dose of from 50 to 1000 mg/day, for example, ribociclib may be administered in a dose of from 50 to 600 mg/day, from 100 to 500 mg/day, from 150 to 450 mg/day or from 200 to 400 mg/day. In some embodiments, the dose is an initial dose for treatment. Pazopanib may be administered in a dose of from 50 to 1000 mg/day, for example, from 250 to 800.

In some embodiments, ribociclib is administered in a dose of from 50 to 1000 mg/day and pazopanib is administered in a dose of from 50 to 1000 mg/day. In some embodiments, ribociclib is administered in a dose of 50 to 600 and pazopanib is administered in a dose of 200 to 800 mg/day.

Any of the solid cancers defined herein may be treatable with a combination of ribociclib and pazopanib, including, but not limited to, melanoma, neuroendocrine tumor, carcinoma (e.g. adenocarcinoma), sarcoma, carcinosarcoma, Ewing sarcoma, liposarcoma, brain tumor, uterus cancer, cervical cancer, head and neck cancer, parotid cancer, salivary gland cancer, adrenal cancer and small bowel cancer.

In some embodiments, a combination comprises abemaciclib and sorafenib for treating solid cancer. According to these embodiments, abemaciclib may be administered in a dose of from 50 to 1000 mg/day, for example, a dose of from 100 to 500 mg/day, from 150 to 450 mg/day, from 200 to 400 mg/day or from 250 to 350 mg/day. In some embodiments, the dose is an initial dose for treatment. Sorafenib may be administered in the dose of from 50 to 1200 mg/day, For example, 250 to 800, from 100 to 900, from 150 to 850, from 200 to 800, or from 300 to 700 mg/day. In some embodiments, abemaciclib is administered in a dose of from 50 to 1000 mg/day and sorafenib is administered in a dose of from 50 to 1200 mg/day. Any of the solid cancers defined herein may be treatable with a combination of abemaciclib and sorafenib, excluding renal cell carcinoma.

In some embodiments, a contemplated combination provides an additive anti-cancer effect.

In some embodiments, a contemplated combination provides synergistic anti-cancer effect. Such a combination is herein defined as a “synergic anti-cancer combination”. In some embodiments, the synergic anti-cancer combination comprises palbociclib and sunitinib, applied for the treatment of any of the solid cancers defined herein, except for hepatocellular carcinoma and appendix cancer. Non-limiting examples of cancers treatable by a synergic anti-cancer combo therapy of palbociclib and sunitinib include cancers characterized by KRAS mutation, for example wherein the KRAS mutation is G12V mutation, cancers characterized by HER2 negative mutation (for example, breast cancer or stomach cancer, which are HER2 negative adenocarcinoma), a breast cancer which is estrogen receptor positive (ER+), a cancer characterized by presence of BRCA1 gene, a cancer that expresses wild-type RAS protein, and adenoid cystic carcinoma (ACC).

In some embodiments, the synergic anti-cancer combination comprises palbociclib and sorafenib, applied for the treatment of any of the solid cancers defined herein, except for hepatocellular carcinoma.

As used herein, the term “synergistic anti-cancer effect” means that the combination of at least one CDK4/4 inhibitor and at least one mtRTKI, as defined herein, exhibits greater anticancer effect or activity than the additive effect or activity provided when each component of the combination is applied alone. The term “synergic” and “synergistic” are used herein interchangeably. A synergistic effect of a combination of therapies permits the use of lower dosages of one or more of the therapeutic agent(s) and/or less frequent administration of the agent(s) to a subject with a disease or disorder, e.g., a proliferative disorder. The ability to lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g., a proliferative disorder. Finally, a synergistic effect of a combination of therapies may avoid or reduce adverse or unwanted side effects associated with the use of either therapeutic agent alone.

In some embodiments, a synergic anti-cancer combination of palbociclib and sunitinib is used for treating multiple types of breast cancer, including, but not limited to, estrogen receptor-positive (ER+) carcinoma, palbociclib resistant carcinoma, ER+ and palbociclib resistant carcinoma, Fluorouracil (FU) resistant carcinoma, HER2 negative carcinoma, triple negative breast cancer (TNBC), i.e., estrogen receptor negative, progesterone receptor negative and HER2 negative, and metastatic cancer.

In some embodiments, a synergic anti-cancer combination of palbociclib and sunitinib is used for treating colon cancer, such as, but not limited to, colon cancer characterized by KRAS mutation, for example, wherein the KRAS mutation is G12V mutation, adenocarcinoma with wild type RAS, and a metastatic adenocarcinoma.

In some embodiments, a synergic anti-cancer combination of palbociclib and sunitinib is used for treating various types of lung cancer including, but not limited to, adenocarcinoma, carcinoma, adenocarcinoma characterized by KRAS mutation, for example, wherein the KRAS mutation is G12V mutation.

In some embodiments, a synergic anti-cancer combination of palbociclib and sunitinib is used for treating various types of ovarian cancer, including, but not limited to, carcinoma, for example, carcinoma characterized by presence of BRCA1 gene, neuroendocrine ovarian cancer, and metastatic neuroendocrine and/or carcinoma.

In some embodiments, a synergic anti-cancer combination of palbociclib and sunitinib is used for treating cholangiocarcinoma.

In some embodiments, a synergic anti-cancer combination comprising palbociclib and sunitinib is used for treating various types of pancreatic cancer, including adenocarcinoma and metastatic adenocarcinoma.

In some embodiments, a synergic anti-cancer combination of palbociclib and sunitinib is used for treating carcinosarcoma.

In some embodiments, a synergic anti-cancer combination of palbociclib and sunitinib is used for treating various types of bones and/or soft tissues cancer, for example, Ewing sarcoma.

In some embodiments, a synergic anti-cancer combination of palbociclib and sunitinib is used for treating adenoid cystic carcinoma.

In some embodiments a synergic anti-cancer combination of palbociclib and sorafenib is used for treating ovarian cancer.

In some embodiments, a synergic anti-cancer combination of palbociclib and sorafenib is used for treating lung cancer.

In some embodiments, the daily administered dose of at least one of the compounds (drugs) of a disclosed combination is lower than the standard daily dose of this compound. For example, the daily administered dose of at least one of the compounds is lower by 5%, 10%, 15%, 20%, 25%, 30%, 35%, 40%, 45%, 50%, 55%, 60%, 65%, 70%, 75%, 80%, 85%, 90%, 95%, 97%, 99%, 99.9% or 99.99% than the standard daily dose of said compound.

For example, when the combination comprises palbociclib and sunitinib, the daily administered dose of palbociclib may be 95%, 90%, 85%, 80%, 75%, 70%, 65%, 60%, 55%, 50%, 45%, 40%, 35%, 30%, 25%, 20%, 15%, or 10% of the standard daily dose of palbociclib. In some embodiments, the daily administered dose of palbociclib is from 90% to 60%, from 80% to 40% or from 60% to 20% of the standard daily dose of palbociclib. The daily administered dose of sunitinib may be 95%, 90%, 85%, 80%, 75%, 70%, 65%, 60%, 55%, 50%, 45%, 40%, 35%, 30%, 25%, 20%, 15% or 10% of the standard daily dose of sunitinib. In some embodiments, the daily administered dose of sunitinib is from 90% to 60%, from 80% to 40% or from 60% to 20% of the standard daily dose of sunitinib. In some embodiments, the daily administered dose of palbociclib is from 90% to 60% of the standard daily dose of palbociclib and the daily administered dose of sunitinib is from 90% to 60% of the standard daily dose of sunitinib.

When the combination is a combination of palbociclib and sorafenib, the daily administered dose of palbociclib may be 95%, 90%, 85%, 80%, 75%, 70%, 65%, 60%, 55%, 50%, 45%, 40%, 35%, 30%, 25%, 20%, 15% or 10% of the standard daily dose of palbociclib. In some embodiments, the daily administered dose of palbociclib is from 90% to 60%, from 80% to 40% or from 60% to 20% of the standard daily dose of palbociclib. The daily administered dose of sorafenib may be 95%, 90%, 85%, 80%, 75%, 70%, 65%, 60%, 55%, 50%, 45%, 40%, 35%, 30%, 25%, 20%, 15% or 10% of the standard daily dose of sorafenib. In some embodiments, the daily administered dose of sorafenib is from 90% to 60%, from 80% to 40% or from 60% to 20% of the standard daily dose of sorafenib. For example, in some embodiments, the daily administered dose of palbociclib is from 90% to 60% of the standard daily dose of palbociclib and the daily administered dose of sorafenib is from 90% to 60% of the standard daily dose of sorafenib.

A synergic combination disclosed herein, for example, a combination of palbociclib and sunitinib or palbociclib and sorafenib, generates much less adverse effect and/or toxic effect that the known chemotaxis drugs used as a standard of care (SOC).

The anti-cancer combinations disclosed herein may be administered by any known route of administration using one of a variety of methods known to those skilled in the art. For example, a combination or a pharmaceutical composition comprising same can be administered, intravenously, arterially, intradermally, intramuscularly, intraperitoneally, subcutaneously, ocularly, sublingually, orally (by ingestion), intranasally (by inhalation), intraspinally, intracerebrally, intraarterially, rectally, topically and/or transdermally (by absorption, e.g., through a skin duct). A combination or a pharmaceutical composition comprising same can appropriately be introduced by rechargeable or biodegradable polymeric devices or other devices, e.g., patches and pumps, or formulations, which provide for the extended, slow or controlled release of the combination or the pharmaceutical composition comprising same. Administration can be performed once daily, a plurality of times during the day and/or over one or more extended periods. In some embodiments, the administration is self-administration. Additionally, or alternatively, administration is by another party who provides a patient with the drug combination. In some embodiments, the combination is orally administered.

The CDK 4/6 inhibitor and the mtRTKI may be administered sequentially (in a sequential manner) and/or in a substantially simultaneous manner (simultaneously). The term “sequential manner” refers to an administration of two or more compounds at different times, and optionally in different modes of administration. The agents can be administered in a sequential manner in either order. The terms “substantially simultaneous manner” refers to administration of two or more compounds with only a short time interval between them. For example, the time interval may be in the range of from 0.01 to 60 minutes. In some embodiments, palbociclib and sunitinib are administered in a sequential manner. In some embodiments, palbociclib and sunitinib are administered in substantially simultaneous manner. In some embodiments, palbociclib and sorafenib are administered in sequential manner. In some embodiments, palbociclib and sorafenib are administered in substantially simultaneous manner.

Each component of a disclosed combination of at least one CDK 4/6 inhibitor and at least one mtRTKI may be formulated as a separate dosage form. For example, in some embodiments, each compound of a combination comprising palbociclib and sunitinib, palbociclib and pazopanib, abemaciclib and sorafenib, abemaciclib and pazopanib, ribociclib and sunitinib, ribociclib and sorafenib or ribociclib and pazopanib, is formulated as a separate dosage form. The dosage form may be a pharmaceutical composition. In some embodiments, each compound is formulated as a separate oral dosage form, e.g. a tablet or a capsule. In some embodiments, each one of the separate dosage forms is administered in a route selected from oral, intravenous (IV) and intramuscular (IM) route of administration.

Alternatively, a disclosed combination is formulated as a single dosage form. In some embodiments, a combination comprising palbociclib and sunitinib, palbociclib and sorafenib, palbociclib and pazopanib, abemaciclib and sorafenib, abemaciclib and pazopanib, abemaciclib and sunitinib, ribociclib and sunitinib, ribociclib and sorafenib or ribociclib and pazopanib is formulated as a single dosage form.

Any of the dosage forms may be formulated or fabricated, for example, as plasters, granules, lotions, liniments, powders, syrups, liquids, solutions, aerosols, extracts, elixirs, ointments, emulsions, suspensions, infusions, tablets, suppositories, injections, spirits, capsules, creams, troches, pastes, pills, and soft or hard gelatin capsules. In some embodiments, the dosage form is formulated or fabricated as tablets.

In one aspect, the present disclosure relates to a kit comprising at least one inhibitor of cyclin-dependent kinase 4 and 6 (CDK 4/6), at least one multi-targeted receptor tyrosine kinase inhibitor (mtRTKI) and instructions for use. In some embodiments, the kit comprises a pharmaceutical composition comprising a CDK 4/6 inhibitor, a pharmaceutical composition comprising an mtRTKI and instructions for use. The CDK 4/6 inhibitor is selected from palbociclib, abemaciclib and ribociclib and the mtRTKI is selected from sunitinib, sorafenib and pazopanib. In some embodiments, the kit does not comprise a combination selected from (i) palbociclib and sorafenib and (ii) abemaciclib and sunitinib. In some embodiments, the kit comprises a pharmaceutical composition comprising palbociclib, optionally in an amount of from 25 to 200 mg, a pharmaceutical composition comprising sunitinib, optionally in an amount of from 5 to 75 mg, and instructions for use. In some embodiments, the kit comprises a pharmaceutical composition comprising palbociclib, optionally in an amount of from 25 to 200 mg, a pharmaceutical composition comprising sorafenib, optionally in an amount of from 100 to 300 mg, and instructions for use. In some embodiments, the kit comprises a pharmaceutical composition comprising from 50 to 600 mg of ribociclib and a pharmaceutical composition comprising 100 to 500 mg of pazopanib. In some embodiments, the kit comprises a pharmaceutical composition comprising from 25 to 300 mg abemaciclib and a pharmaceutical composition comprising 100 to 300 mg of sorafenib. The kit is for use in treating any of the solid cancers defined herein. In some embodiments, the cancer is not a hepatocellular carcinoma. In some embodiments, the cancer is not renal cell carcinoma. In some embodiments, the cancer is not appendix carcinoma. In some embodiments, use of the kit provides a synergistic anticancer effect.

In another aspect, the present disclosure relates to a pharmaceutical composition comprising at least one inhibitor of cyclin-dependent kinase 4/6 (CDK 4/6) and at least one multi-targeted receptor tyrosine kinase inhibitor (mtRTKI), and a pharmaceutically acceptable excipient. The at least one CDK 4/6 inhibitor may be palbociclib, abemaciclib or ribociclib. Theat least one mtRTKI may be sunitinib, sorafenib or pazopanib.

The terms “pharmaceutical composition” and “formulation”, as used herein, are interchangeable and refer to a composition comprising a compound, or a combination of compounds disclosed herein formulated together with one or more pharmaceutically acceptable carriers and/or excipients.

The terms “pharmaceutically acceptable carrier and “pharmaceutically acceptable excipient”, refer to any and all solvents, dispersion media, preservatives, antioxidants, coatings, isotonic agents, absorption delaying agents, surfactants, fillers, disintegrants, binders, diluents, lubricants, glidants, pH adjusting agents, buffering agents, enhancers, wetting agents, solubilizing agents, and the like, that are compatible with pharmaceutical administration.

The contemplated formulations can be produced by known methods using conventional solid carriers or excipients such as, for example, lactose, starch or talcum or liquid carriers such as, for example, water, fatty oils or liquid paraffin. Other carriers or excipients which may be used include, but are not limited to, materials derived from animal or vegetable proteins; gums such as acacia, guar, agar, and xanthan; polysaccharides; alginates; carboxymethyl celluloses; carrageenan; dextran; pectin; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminum silicates; and amino acids having from 2 to 12 carbon atoms and derivatives thereof such as, but not limited to, glycine, L-alanine, L-aspartic acid, L-glutamic acid, L-hydroxyproline, L-isoleucine, L-leucine and L-phenylalanine.

Auxiliary components such as tablet disintegrants, solubilizes, preservatives, antioxidants, surfactants, viscosity enhancers, coloring agents, flavoring agents, pH modifiers, sweeteners or taste-masking agents may also be incorporated into a contemplated pharmaceutical composition. In some embodiments, the pharmaceutical composition is particularly suitable for oral administration. It is contemplated that by orally administering the composition, a systemic effect can be achieved.

A contemplated pharmaceutical composition may comprise at least the following combinations: palbociclib and sunitinib; palbociclib and sorafenib, palbociclib and pazopanib; abemaciclib and sorafenib; abemaciclib and pazopanib; ribociclib and sunitinib; abemaciclib and sunitinib, ribociclib and sorafenib; and ribociclib and pazopanib. In some embodiments, any one of these pharmaceutical compositions is in the form of a tablet or a capsule.

In some embodiments, a disclosed pharmaceutical composition comprises palbociclib and sunitinib. The amount of palbociclib may be from 25 to 250 mg, and the amount of sunitinib may be from 5 to 75 mg, for example, from 25 to 250, from 30 to 225, from 35 to 200, from 40 to 175, from 50 to 150, from 55 to 130, from 60 to 125 or from 75 to 100 mg of palbociclib, and from 10 to 70, from 15 to 65, from 20 to 60, from 20 to 60, from 25 to 55, from 30 to 50 or from 35 to 45 mg of sunitinib.

In some embodiments, a disclosed pharmaceutical composition comprises palbociclib and sorafenib. The amount of palbociclib may be from 25 to 200 mg and the amount of sorafenib may be from 100 to 300 mg, for example, from 25 to 250, from 30 to 225, from 35 to 200, from 40 to 175, from 50 to 150, from 55 to 130, from 60 to 125 or from 75 to 100 mg of palbociclib, and from 110 to 290, from 120 to 280, from 130 to 260, from 150 to 240, from 180 to 220, from 190 to 210, from 100 to 200 or from 200 to 300 mg of sorafenib.

In some embodiments, a contemplated pharmaceutical composition comprises ribociclib and pazopanib. The amount of ribociclib may be from 50 to 600 mg and the amount of pazopanib may be from 100 to 500 mg, for example, from 50 to 600 mg, from 75 to 575, from 100 to 550, from 125 to 525, from 150 to 500, from 200 to 450, from 250 to 400 or from 300 to 350 of ribociclib, and from 150 to 450, from 200 to 400 or from 250 to 350 mg of pazopanib.

In embodiment, a contemplated pharmaceutical composition comprises abemaciclib and sorafenib. The amount of abemaciclib may be from 25 to 300 mg and the amount of sorafenib may be from 100 to 300 mg, for example, from 110 to 290, from 120 to 280, from 130 to 260, from 150 to 240, from 180 to 220, from 190 to 210, from 100 to 200 or from 200 to 300 mg of sorafenib, and from 30 to 290, from 50 to 280, from 100 to 260, from 150 to 240, from 180 to 220, from 190 to 210, from 100 to 200 or from 200 to 300 mg of abemaciclib

According to any one of the above embodiments, a pharmaceutical composition that comprises any of the combinations defined above provides a synergic therapeutic effect when used in treating a solid cancer as defined herein, thus, it is referred to herein as a “synergic pharmaceutical composition”.

In some embodiments, a disclosed pharmaceutical composition comprises a combination of palbociclib and sunitinib as defined herein, for the treatment of ACC.

In a further aspect, the present disclosure relates to a method of treating solid cancer in a subject in need thereof, comprising co-administering to the subject at least one inhibitor of cyclin-dependent kinase 4/6 (CDK 4/6) selected from palbociclib, abemaciclib and ribociclib, and at least one multi-targeted receptor tyrosine kinase inhibitor (mtRTKI) selected from sunitinib, sorafenib and pazopanib. In some embodiments, the cancer is not hepatocellular carcinoma and/or not a renal cell carcinoma. In some embodiments, the combination is not a combination of (i) palbociclib and sorafenib or (ii) abemaciclib and sunitinib.

In some embodiments, the treatment provides a synergistic effect.

Some embodiments, of the present disclosure relate to a method of treating solid cancer in a subject in need thereof, comprising administering to the subject a combination of palbociclib and sunitinib as defied herein, wherein the method provides a synergic anticancer effect, provided that the cancer is not hepatocellular carcinoma or appendix cancer. Other embodiments relate to a method of treating solid cancer in a subject in need thereof, comprising administering palbociclib and sorafenib, wherein the method provides a synergic anticancer effect, provided that the cancer is not hepatocellular carcinoma. Non-limiting examples of solid cancers treatable by a disclosed method include melanoma, neuroendocrine tumor, carcinoma, Ewing sarcoma, carcinosarcoma, sarcoma, lung cancer, stomach cancer, breast cancer, ovarian cancer, colon cancer, neuroendocrine cancer, pancreas cancer, cholangiocarcinoma, bone cancer, liposarcoma, and adrenal cancer. In some embodiments, the cancer is ACC.

In yet another aspect, the present disclosure relates to the use of at least one CDK 4/6 inhibitor selected from palbociclib, abemaciclib and ribociclib and at least one mtRTKI selected from sunitinib, sorafenib and pazopanib for preparation of a medicament for treating solid cancer. In some embodiment, cancer is not hepatocellular carcinoma or renal cell carcinoma. In some embodiments, a combination of palbociclib and sunitinib is used for preparation of a medicament for treating solid cancer. In some embodiments, a combination of palbociclib and sorafenib is used for preparation of a medicament for treating solid cancer.

The term “co-administration” as used herein has the meaning of administering two or more compounds in a regimen selected from a single combined composition (single dosage form), separate individual compositions (multiple dosage forms) administered substantially at the same time, and separate individual compositions administered under separate schedules and include treatment regimens in which the compounds are not necessarily administered by the same route of administration and/or at the same time. According to some embodiments, the term “co-administration” encompasses administration of a first and second compound in an essentially simultaneous manner, such as in a single dosage form, e.g., a capsule or tablet having a fixed ratio of first and second amounts, or in multiple dosage forms for each. The agents can be administered in a sequential manner in either order.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value

[symbol]

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Having now generally described embodiments of the present disclosure, the same will be more readily understood through reference to the following examples, which are provided by way of illustration and are not intended to be limiting of the present invention.

### EXAMPLES

### Materials and Methods

Cancer specimens were obtained from core needle biopsy, tumor resection, bone-marrow (BM) aspiration biopsies, ascites and/or pleural effusion of patients diagnosed and treated at the Davidoff center, Rabin medical center of Israel.

All mice were maintained and treated in accordance with the Rabin medical center guide for the care and use of experimental animals with approval from the RMC Institutional Animal Care and Use Committee.

Tumor materials were placed in cold DMEM medium supplemented with 10% FBS and 1:100 penicillin/streptomycin antibiotics and maintained on ice until processing. Within 0.5-2 hours, tumor fragments were cut into small pieces (approximately 2-3 mm) using sterile surgical instruments. Several pieces were used for implantation and the remaining pieces were preserved in 10% DMSO/90% FCS freezing medium, snap-frozen in liquid nitrogen, and another piece was later histological examination. formalin-fixed for Typically, several implantations were carried out: subcutaneously on the flanks, intraperitoneal implantation, and implantation directly into the mammary of recipient 5-8 weeks old female immunodeficient NRG or NSG mice (Jackson Laboratories: NSG is NOD.Cg-PrkdcscidlI2rgtm1Wjl/SzJlI strain, and NRG is NOD.Cg-Rag1tm1Mom lI2rgtm1Wjl/SzJ strain). For samples obtained from breast cancer patients, mice were also supplemented with 17β-estradiol (Tocris, Cas #50-28-2) using slow release by osmotic pumps implanted subcutaneously on the back (28-day release, 1.08 mg/pellet, Alzet®). Before implantation, the tumor fragments were coated with Cultrex™ Basement membrane matrix, type3 (Trevigen®). For samples from either pleural effusion, ascites: ascites/pleural fluid was centrifuged at 1500 RPM for 4 min, washed with PBS and cells were counted. For BM samples: mononuclear cells (MNCs) were isolated using Ficoll® (Sigma), and cells were counted. About 2-3 million cells resuspended in a volume of 0.05-0.1 ml Cultrex™:PBS(1:1), were injected subcutaneously (SC), intraperitoneally (IP), or intravenously (IV). In some cases of BM samples, injection was done from the total BM aspiration without any manipulation. Surgery was performed under sterile conditions in a laminar flow cabinet using sterilized surgical instruments. Mice were kept under pathogen-free conditions and received sterilized food and water ad libitum.

### Tumor Growth

Mice were weighted and inspected 1-2/week for assessment of general condition, and patient-derived xenografts (PDX) development was assessed by palpation of the site of implantation and measured in two dimensions by electronic caliper. Tumor volume was determined by the ellipsoidal formula (width×width×length)/2. Once tumors reached 1-1.5 cm in diameter, mice were euthanized, and tumors were harvested. Tumor tissue was passaged directly into further generation or for storage.

Pathological assessment of tumors: Paraffin blocks (formalin-fixed paraffin-embedded; FFPE) were prepared from tumor sections preserved in 4% paraformaldehyde (PFA) and slides were stained with Hematoxylin & Eosin (H&E) solution for initial histopathological evaluation. Histopathological examination confirmed their human origin and their morphological similarity to the corresponding engrafted tumor.

### Drug Efficacy Experiments

Fresh tumors were excised from mice, dissociated by gentle MACS™ dissociator and implanted by subcutaneous injection into the neck of NRG mice. Mice were at age of 7-12 weeks; gender of the mice were in accordance with patient's gender. When tumors reached a size of 60-200 mm³, mice were assigned to the various treatment groups based on the tumor size, tumor growth rate and body weight (BW), creating groups with similar averages of these parameters. Drugs dosing was initiated at the same day of randomization, denoted as Day 0. Mice were treated with vehicle, or with drugs as described in the examples below. All doses were delivered by oral gavage in a total volume of 100 μL/28 gr. Drugs were administered daily for 5 days/week

Tumor volume and BW were measured twice a week throughout the treatment period. When tumor volume reached 1500 mm³, mice were euthanized according to IACUC approved protocol. The tumors were surgically removed and stored (fixed at 4% PFA followed by FFPE, and frozen in liquid-nitrogen).

Survival curves were calculated using Kaplan-Meier approach, and comparison of the survival curves was done using log rank test (Kuhfeld and So, “Creating and Customizing the Kaplan-Meier Survival Plot in PROC LIFETEST”, SAS Global Forum 2013, SAS Institute Inc.).

### Statistical Analysis

Statistics calculations were performed by a third independent party. All measured variables and derived parameters were tabulated by descriptive statistics.

The experiments were performed using constant drug doses and the comparison between the groups was made on the last day in which all the treatment groups of Drug A, Drug B, and combination of A+B were available.

A direct method to determine synergy was applied, including comparing the drugs combination (A+B) effect to the effect of each drug separately (A, B), as follows.

The non-parametric Wilcoxon-Mann-Whitney Rank sum test for independent samples was applied for testing the statistical significance of the difference between treatment groups for the primary endpoint (tumor volume) per experiment and time. P-value of 0.05 or lower was considered statistically significant.

To prove the synergic effect, as a first step, the superiority of the drugs combination over each drug's effect (when administered alone) must be statistically significant. In a second step, if there is no statistically significant difference between effects of drugs A and B, each alone, an average of the two effects is calculated to obtain a more precise estimate of the effect. If effects of A and B are different, the average has no statistical value.

The superiority of the combination over the average effect (of A and B, each alone) must be statistically significant, provided that the effect of drugs A and B are not statistically different.

If the effect of the combination is higher than the effect of each one of the separate treatments with statistical significance, and the effect of the combination is higher than the average effect of the two treatments with statistical significance (if there is no statistically significant difference between effects of two drugs when administered alone), then synergy is proved. Otherwise, additive effect is concluded.

Survival analysis using Kaplan-Meier survival function curve was applied for testing the statistical significance of the difference in overall survival between treatment groups per experiment, and for all experiments pooled together. The Log-Rank test was used for treatment comparison

All tests were two-tailed, and a p value of 5% or less was considered statistically significant.

### Example 1A. Efficacy of the Palbociclib+Sunitinib Combo Treatment of Colon Cancer

Colon cancer tissue (adenocarcinoma comprising KRAS mutation G12V), obtained by an ultrasound directed liver biopsy from a liver metastatic lesion, was collected and grown in NRG mice. Then the tumor tissue was collected, dissociated and implanted in new NRG mice, and when reached a size of 60-110 mm³, mice were assigned to treatment groups as described in Table 1 (PO: per os, i.e., by mouth; IP: intraperitoneal).

TABLE 1
Study Design

| | | | # of treatments | Treatment | # of mice |
|---|---|---|---|---|---|
| Group # | Tested drugs | | per week | method | per group |
| 1 | control (vehicle) | | 5 | PO | 5 |
| 2 | palbociclib 100 | mg/kg | 5 | PO | 6 |
| 3 | sunitinib 50 | mg/kg | 5 | PO | 5 |
| 4 | palbociclib 100 | mg/kg + | 5 | PO | 6 |
| | sunitinib 50 | mg/kg | | | |
| 5 | gemcitabine 25 | mg/kg | 2 | IP | 5 |

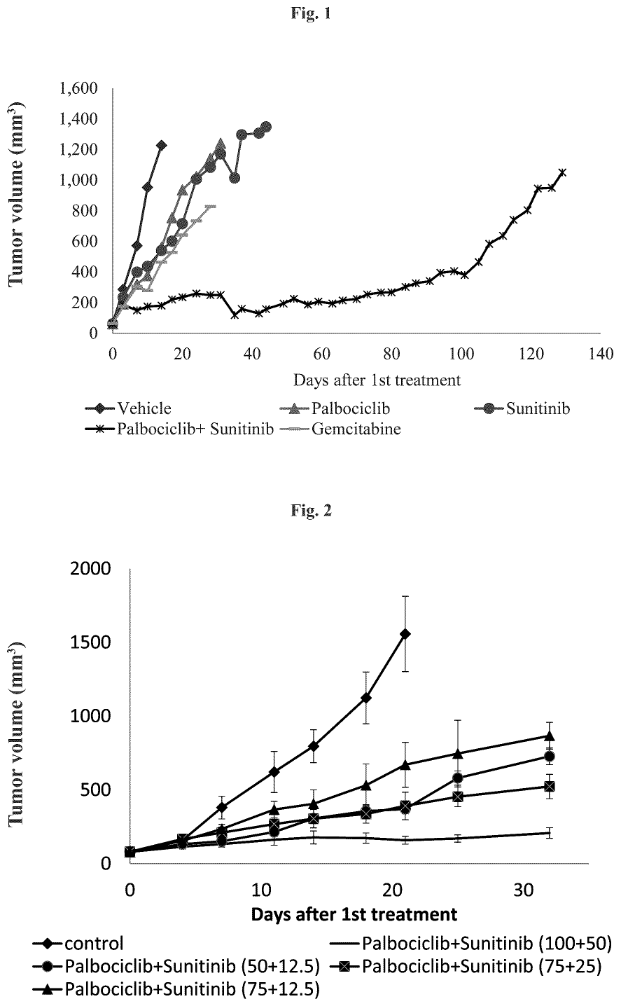

The experiment lasted 129 days. Vehicle was used as a negative control and gemcitabine (a known anticancer drug) was used as a standard of care (SOC) treatment. The results, showing effects of palbociclib, sunitinib and their combination on tumor volume, are presented in FIG. 1. It can be clearly seen from FIG. 1 that, whereas cancer in the presence each of the anticancer drugs alone quickly progressed, concomitant administration of palbociclib and sunitinib significantly and effectively inhibited growth of the tumor for more than 70 days. It seems that after about 60 days of tumor growth inhibition in the combo group, tumors start to develop resistance to therapy and re-grow. After >100 days of treatment in the combo group, 2 mice developed ulcers. As seen in FIG. 1, the combination was much more effective than the standard treatment with gemcitabine. The synergic effect of palbociclib and sunitinib combination was statistically significant as seen in the P-values of Wilcoxon Test presented in Table 2 and in the log-rank test of overall survival testing the combination of palbociclib and sunitinib vs each one of these drugs alone (Table 3).

TABLE 2
Wilcoxon Test P-values

| | | P-value, |
|---|---|---|
| | | Wilcoxon |
| | | Test |
| Treatment | Day | (Two-sided) |
| Palbociclib vs combo | 31 | 0.0081 |
| Sunitinib vs combo | 31 | 0.0122 |
| Mean Palbociclib | 31 | 0.0081 |
| Sunitinib vs combo | | |

TABLE 3
Log-rank values

| Treatment | Log-Rank |
|---|---|
| Palbociclib vs combo | 0.0099 |
| Sunitinib vs combo | 0.0494 |

It can be clearly seen that the combination of palbociclib+sunitinib had a synergetic inhibitory effect compared to the effect of palbociclib or sunitinib, each alone, in treatment of colon cancer.

Further, the effect of drugs doses on tumor volume was tested for 32 days. Mice were allocated to treatment as defined in Table 4 (PO: per os; IP: intraperitoneal).

TABLE 3
Study Design

| | | | # of treatments | Treatment | # of mice |
|---|---|---|---|---|---|
| Group # | Tested drugs | | per week | method | per group |
| 1 | Control (vehicle) | | 5 | PO | 5 |
| 2 | palbociclib 100 | mg/kg + | 5 | PO | 6 |
| | sunitinib 50 | mg/kg | | | |
| 3 | palbociclib 50 | mg/kg + | 5 | PO | 5 |
| | sunitinib 12.5 | mg/kg | | | |
| 4 | palbociclib 75 | mg/kg + | 5 | PO | 6 |
| | sunitinib 25 | mg/kg | | | |
| 5 | palbociclib 75 | mg/kg + | 5 | IP | 6 |
| | sunitinib 12.5 | mg/kg | | | |

The results are presented in FIG. 2. As can clearly be seen from the figure, the response of colon tumor to the combo treatment with palbociclib and sunitinib was dose dependent. Administrating of 2-fold higher doses of both drugs was shown to synergically suppress tumor growth in a dose-response manner.

### Example 1B. Efficacy of the Palbociclib+Sunitinib Combo Treatment of Colon Cancer

In an additional experimental arrangement, colon tumor (well differentiated adenocarcinoma) was grown in NRG mice, then collected, dissociated and implanted in new NRG mice and when reached of 60-90 mm³, mice were assigned to treatment groups as described in Table 5 (PO: per os; IP: intraperitoneal).

TABLE 5
Study Design

| | | | # of treatments | Treatment | # of mice |
|---|---|---|---|---|---|
| Group # | Tested drugs | | per week | method | per group |
| 1 | Control (vehicle) | | 5 | PO | 5 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 5 |
| | Sunitinib 50 | mg/kg | | | |
| 5 | 5-FU 30 | mg/kg + | 2 | IP | 5 |
| | Irinotecan 20 | mg/kg | | | |

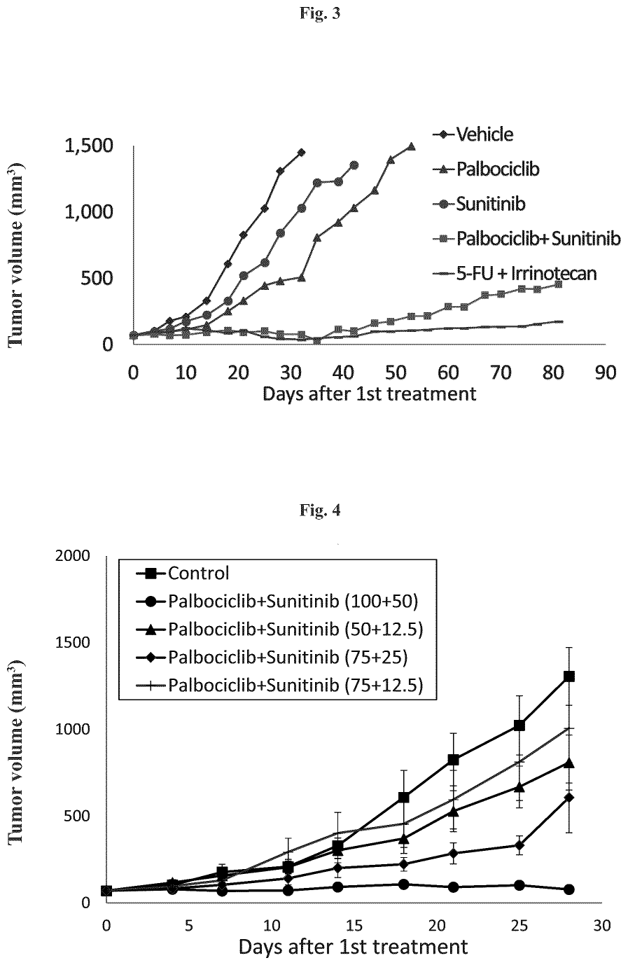

The experiment lasted 81 days. Vehicle was used as a negative control and administration of the chemotherapy drugs fluorouracil (5-FU) and Irinotecan as SOC treatment (positive control). The results showing effects of palbociclib, sunitinib and their combination on tumor volume size are presented in FIG. 3. Table 6 summarizes the P-values of Wilcoxon Test. Within the arrangement of the experiment, it was also possible to calculate Log-rank values, which are presented in Table 7.

FIG. 3 shows that palbociclib alone attenuated tumor growth and sunitinib treatment had little or almost no effect on tumor volume. It can be clearly seen from FIG. 3 that whereas cancer in the presence of each one of the anticancer drugs alone quickly progressed, concomitant administration of palbociclib and sunitinib (combo treatment) significantly and effectively inhibited growth of the tumor for more than 80 days. After about 60 days of tumor growth inhibition in the combo group, tumors started to develop resistance to therapy and re-grew. It is noted that the combo treatment had similar effect as the standard treatment with 5FU and Irinotecan at least up to 60 days.

TABLE 6
Wilcoxon Test P-values

| | | P-value, |
|---|---|---|
| | | Wilcoxon |
| | | Test |
| | | (Two- |
| Treatment | Day | sided) |
| palbociclib vs combo | 42 | 0.0122 |
| sunitinib vs combo | 42 | 0.0122 |
| Mean palbociclib | 42 | 0.0122 |
| sunitinib vs combo | | |

TABLE 7
Log-rank values.

| Treatment | Log-Rank |
|---|---|
| palbociclib vs combo | 0.0144 |
| sunitinib vs combo | 0.0345 |

All these data indicate that the combination of palbociclib with sunitinib synergically inhibited growth of colon cancer tumor.

The effect of various doses of palbociclib and sunitinib administered in combination (combo treatment) on tumor volume was tested in a model of colon cancer. The experiment was performed for 28 days, and the mice were allocated according to Table 8. The results are presented in FIG. 4.

TABLE 8
Study Design

| | | | # of treatments | Treatment | # of mice |
|---|---|---|---|---|---|
| Group # | Tested drugs | | per week | method | per group |
| 1 | Control (vehicle) | | 5 | PO | 5 |
| 2 | Palbociclib 100 | mg/kg + | 5 | PO | 5 |
| | Sunitinib 50 | mg/kg | | | |
| 3 | Palbociclib 50 | mg/kg + | 5 | PO | 5 |
| | Sunitinib 12.5 | mg/kg | | | |
| 4 | Palbociclib 75 | mg/kg + | 5 | PO | 5 |
| | Sunitinib 25 | mg/kg | | | |
| 5 | Palbociclib 75 | mg/kg + | 5 | PO | 5 |
| | Sunitinib 12.5 | mg/kg | | | |

A clear dose-dependent response was observed for the combination. Administrating of 2-fold higher doses of both drugs was shown to synergically suppress tumor growth in a dose-response manner.

### Example 1C. Efficacy of the Palbociclib+Sunitinib Combo Treatment of Colon Cancer

In a further experimental arrangement, colon tumor (metastatic adenocarcinoma with wild-type RAS) was grown in NRG mice, then collected, dissociated and implanted in new NRG mice, and when reached a size of 60-170 mm³, mice were assigned to treatment groups as described in Table 9 (PO: per os; IP: intraperitoneal).

TABLE 9
Study Design

| | | | # of treatments | Treatment | # of mice |
|---|---|---|---|---|---|
| Group # | Tested drugs | | per week | method | per group |
| 1 | Control (vehicle) | | 5 | PO | 5 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 5 |
| | Sunitinib 50 | mg/kg | | | |
| 5 | Folfox | | 1 | IP | 6 |
| | (leucovorin 70 | mg/kg + | | | |
| | oxaliplatin 4 | mg/kg + | | | |
| | 5FU 30 | mg/kg) | | | |
| | Avastin 5 | mg/kg | 2 | | |

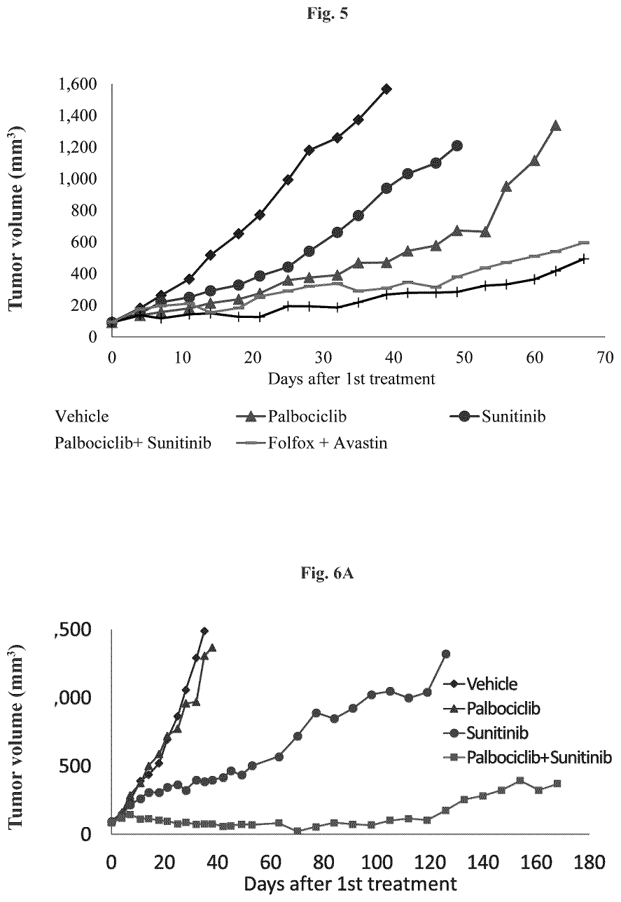

The experiment lasted 67 days. Vehicle used as a negative control and administration of Folfox+avasting as was used SOC treatment. This SOC combination chemotherapy regimen that is used to treat colorectal cancer includes the drugs leucovorin calcium (folinic acid), fluorouracil, and oxaliplatin. The results showing the effect of palbociclib, sunitinib and their combination on tumor volume are presented in FIG. 5. Table 10 summarizes the P-values of Wilcoxon Test.

FIG. 5 shows that palbociclib alone attenuated tumor growth and sunitinib treatment had only moderate effect on tumor volume. It can be clearly seen from FIG. 5 that whereas cancer in the presence of each one of the anticancer drugs alone kept progressing, concomitant administration of palbociclib and sunitinib significantly and effectively inhibited growth of the tumors for more than 60 days. Moreover, while at day 52 the tumors developed resistance to palbociclib (at this point tumors in mice treated with sunitinib reached maximal size defined for euthanization of mice), the combo treatment successfully inhibited the progression of cancer. This clearly indicates the synergy between palbociclib and sunitinib. In addition, the combination had a long-lasting effect of tumor inhibition, similar to the standard treatment. Moreover, the combination was significantly safer for the mice than the SOC treatment. On day 18, 2 out of 5 mice in the SOC group were excluded from the experiment due to toxicity with severe clinical signs (weakness and loss of weight), compared to the combination group in which all 5 mice were treated without any toxicity signs until day 67.

TABLE 10
Wilcoxon Test P-values

| | | P-value, |
|---|---|---|
| | | Wilcoxon |
| | | Test |
| Treatment | Day | (Two-sided) |
| Palbociclib vs combo | 49 | 0.0216 |
| Sunitinib vs combo | 49 | 0.0216 |
| Mean Palbociclib | 49 | 0.0122 |
| Sunitinib vs combo | | |

### Example 2. Efficacy of the Palbociclib+Sunitinib, Ribociclib+Pazopanib and Abemaciclib+Sorafenib Combo Treatments of Stomach Cancer

Stomach cancer tissue (poorly differentiated stomach adenocarcinoma (HER2 negative)), obtained by a biopsy was grown in NRG mice, then collected, dissociated and implanted in new NRG mice for drug efficacy study. When tumors reached a size of 60-160 mm³, mice were assigned to treatment groups based on the tumor size, tumor growth rate and body weight (BW), creating groups with similar averages of these parameters (see Table 11). Drugs dosing was initiated at the same day of randomization, denoted as Day 0 (PO: per os; IP: intraperitoneal).

TABLE 11
Study Design

| | | | # of | | # of |
|---|---|---|---|---|---|
| | | | treatments | | mice |
| Group | | | per | Treatment | per |
| # | Tested drugs | | week | method | group |
| 1 | Control (vehicle) | | 5 | PO | 16 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 6 |
| | Sunitinib 50 | mg/kg | | | |
| 5 | Ribociclib 100 | mg/kg | 5 | PO | 4 |
| 6 | Pazopanib 100 | mg/kg | 5 | PO | 4 |
| 7 | Ribociclib 100 | mg/kg + | | PO | 5 |
| | Pazopanib 100 | mg/kg | | | |
| 8 | Abemaciclib 100 | mg/kg | 5 | PO | 4 |
| 9 | Sorafenib 20 | mg/kg | 5 | PO | 4 |
| 10 | Abemaciclib 100 | mg/kg + | | PO | 5 |
| | Sorafenib 20 | mg/kg | | | |
| 11 | Oxaliplatin 4 | mg/kg + | 2 | IP | 5 |
| | 5-FU 30 | mg/kg | | | |

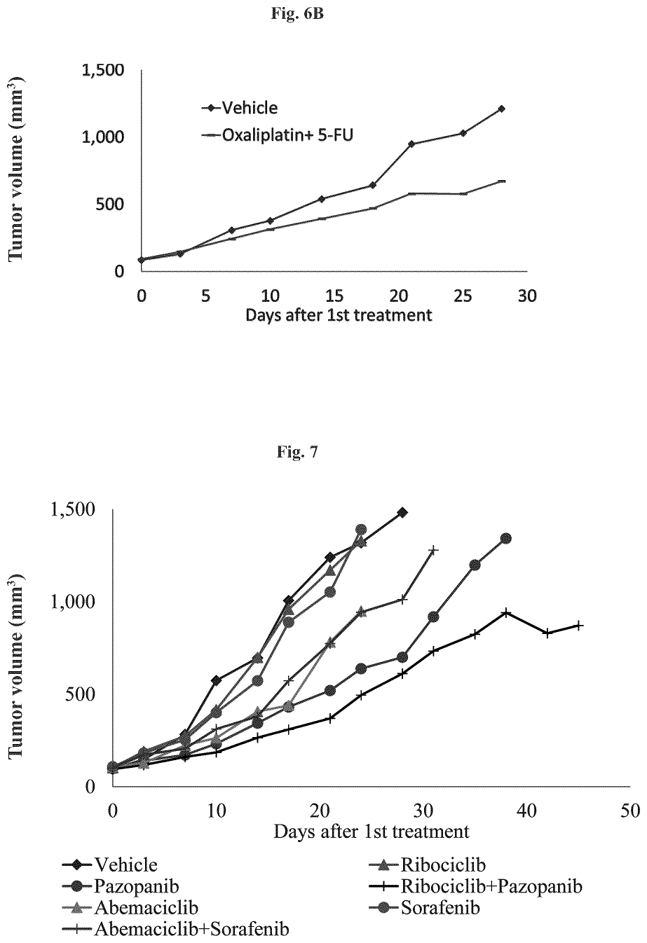

The results are presented in FIGS. 6-7.

As clearly seen in FIG. 6A, a combination of palbociclib and sunitinib provided a much stronger effect than would have been expected from combining the effects of each one of these drugs administered alone. Moreover, the combination provided a long-lasting effect of a complete inhibition of tumor development until day 120, and inhibited tumor growth for additional 40 days (until the end of the experiment). On day 126, two mice showed complete regression and one mouse had a stable disease (tumor volume of 80 mm³). After ˜120 days of tumor growth inhibition in the combo group, tumors started to develop resistance to therapy and re-grew. After >100 days of treatment in the combo group, 2 mice developed ulcers (in the tumors) and were removed from the study.

The effect of SOC treatment with a combination of oxaliplatin+5-FU on tumor volume is shown in FIG. 6B. It can be seen, when comparing FIG. 6A and 6B, that the combo treatment was much more efficient than the standard treatment with oxaliplatin+5FU.

FIG. 7 shows the effects of further combinations of CDK4/6 inhibitors and mtRTKIs on tumor volume: ribociclib+pazopanib and abemaciclib+sorafenib. As seen in the figure, not all combinations of drugs were as effective as a combination of palbociclib and sunitinib. For example, a combination of ribociclib and pazopanib was not more effective than pazopanib alone. A combination of abemaciclib and sorafenib was as effective as abemaciclib alone. As already discussed, the effect of a combinatory treatment cannot be predicted a priori and, moreover, one cannot predict whether a combination will have a synergic effect, as in the case of a combination of palbociclib and sunitinib.

The results above were statistically significant, as indicated in Table 12, showing P-values of Wilcoxon Test for all combinations. The statistical analysis supported the conclusion that combination of palbociclib and sunitinib was synergistic in treatment of stomach cancer, whereas no synergy was obtained for the other combination tested.

TABLE 12
Wilcoxon Test P-values

| | | P-value, |
|---|---|---|
| | | Wilcoxon |
| | | Test |
| | | (Two- |
| Treatment | Day | sided) |
| Palbociclib vs combo | 38 | 0.0081 |
| Sunitinib vs combo | 38 | 0.0081 |
| Mean Palbociclib Sunitinib | 38 | 0.0081 |
| vs combo | | |
| Sunitinib vs combo | 126 | 0.0200 |
| Ribociclib vs combo 2 | 24 | 0.0200 |
| Pazopanib vs combo 2 | 24 | 0.3913 |
| Mean Ribociclib | 24 | 0.0662 |
| Pazopanib vs combo 2 | | |
| Abemaciclib vs combo 3 | 24 | 1.0000 |
| Sorafenib vs combo 3 | 24 | 0.2703 |
| Mean Abemaciclib | 24 | 0.7133 |
| Sorafenib vs combo 3 | | |

### Example 3A. Efficacy of the Palbociclib+Sunitinib and Ribociclib+Pazopanib Combo Treatments of Breast Cancer

Invasive ductal carcinoma (ER positive) that was clinically shown to be resistant to palbociclib, was grown in NRG mice, then collected, dissociated and implanted in new NRG mice for drug efficacy study. When tumors reached a size of 60-180 mm³, mice were assigned to treatment groups based on the tumor size, tumor growth rate and body weight (BW), creating groups with similar averages of these parameters (Table 13). Drugs dosing was initiated at the same day of randomization, denoted as Day 0. Tumor volume was measured (PO: per os).

TABLE 13
Study design

| | | | # of treatments | Treatment | # of mice |
|---|---|---|---|---|---|
| Group # | Tested drugs | | per week | method | per group |
| 1 | Control (vehicle) | | 5 | PO | 5 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 6 |
| | Sunitinib 50 | mg/kg | | | |
| 5 | Ribociclib 100 | mg/kg | 5 | PO | 5 |
| 6 | Pazopanib 100 | mg/kg | 5 | PO | 5 |
| 7 | Ribociclib 100 | mg/kg + | 5 | PO | 5 |
| | Pazopanib 100 | mg/kg | | | |

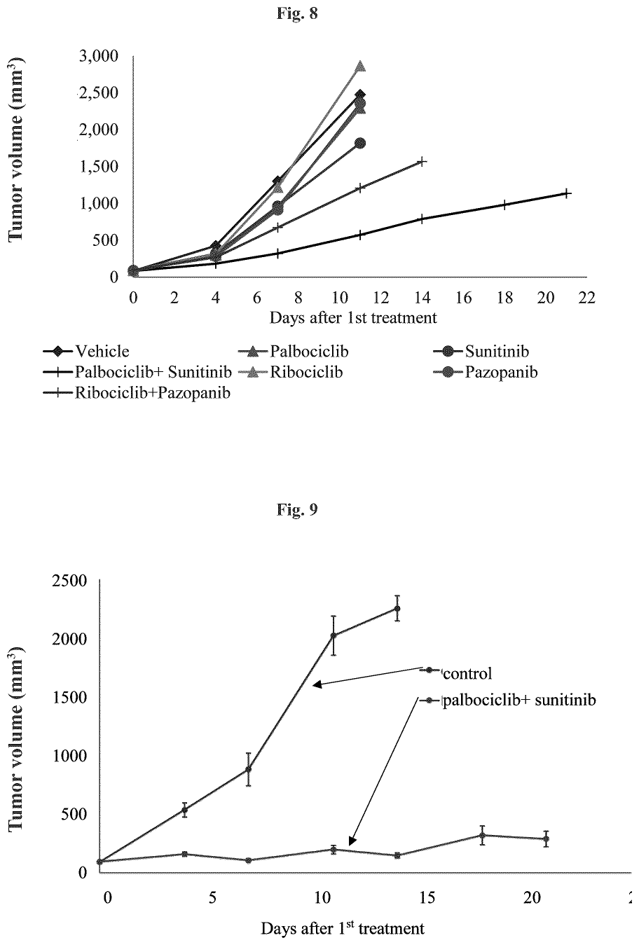

The results are presented in FIG. 8. As can be seen, each one of the drugs, i.e., palbociclib, sunitinib, ribociclib and pazopanib, when administered as monotherapies, had weak to mild effect on tumor size. However, combination of ribociclib and pazopanib showed an additive effect, whereas the combination of palbociclib and sunitinib showed a synergic effect in breast cancer. The synergic effect was statistically significant as followed from P-values of Wilcoxon Test (Table 14) and further supported by log-rank values of survival test (Table 15).

TABLE 14
Wilcoxon Test P-values

| | | P-value, Wilcoxon |
|---|---|---|
| Treatment | Day | Test (Two-sided) |
| Palbociclib vs combo | 11 | 0.0137 |
| Sunitinib vs combo | 11 | 0.0137 |
| Mean Palbociclib | 11 | 0.0137 |
| Sunitinib vs combo | | |
| Ribociclib vs combo | 11 | 0.0122 |
| Pazopanib vs combo | 11 | 0.1779 |
| Mean Ribociclib | 11 | 0.0601 |
| Pazopanib vs combo | | |

TABLE 15

| Treatment | Log-Rank |
|---|---|
| Palbociclib vs combo | 0.0088 |
| Sunitinib vs combo | 0.0091 |

### Example 3B. Efficacy of the Palbociclib+Sunitinib Combo Treatment of Breast Cancer

Breast cancer cells of cancer that was clinically shown to be resistant to palbociclib (data not shown) were collected by bone marrow aspiration and grown in immunocompromised mice. Then, the grown tumor mass was collected, dissociated and implanted subcutaneously into NRG mice necks as described in Material and Methods. First, the mice were treated with fluorouracil (5-FU) (30 mg/kg, IP 2/week), with variable attenuation of tumor growth between mice. One of the tumors was taken for further implantation to study the effect of a combination therapy comprising palbociclib+sunitinib. The implanted mice were treated orally with combo treatment (6 mice) or not treated (control; 6 mice) 5 times/week for 20 days. The results are presented in FIG. 9.

As seen in FIG. 9, tumors resistant to 5-FU treatment were effectively treated with combination of palbociclib+sunitinib. This treatment completely prevented development and growth of 5-FU resistant breast cancer. Clearly, the combo treatment was much more efficient that would have been expected. In fact, combo treatment completely prevented development of 5FU resistant breast cancer.

### Example 3C. Efficacy of the Palbociclib+Sunitinib and Ribociclib+Pazopanib Combo Treatments of Breast Cancer

Triple negative breast cancer (TNBC) tissue was grown in NRG mice, then tumors were collected, dissociated and implanted in new NRG mice for drug efficacy study. When tumors reached a size of 60-210 mm³, mice were assigned to treatment groups based on the tumor size, tumor growth rate and body weight, creating groups with similar averages of these parameters (Table 16). Drugs dosing was initiated at the same day of randomization, denoted as Day 0. The experiment lasted 45 days. Tumor volume was measured (PO: per os; IP: intraperitoneal).

TABLE 16
Study design

| Group | | | # of treatments | Treatment | # of mice |
|---|---|---|---|---|---|
| # | Tested drugs | | per week | method | per group |
| 1 | Control (vehicle) | | 5 | PO | 6 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 5 |
| | Sunitinib 50 | mg/kg | | | |
| 5 | Ribociclib 100 | mg/kg | 5 | PO | 5 |
| 6 | Pazopanib 100 | mg/kg | 5 | PO | 5 |
| 7 | Ribociclib 100 | mg/kg + | 5 | PO | 5 |
| | Pazopanib 100 | mg/kg | | | |
| 8 | Cisplatin 3 | mg/kg | 2 | IP | 4 |

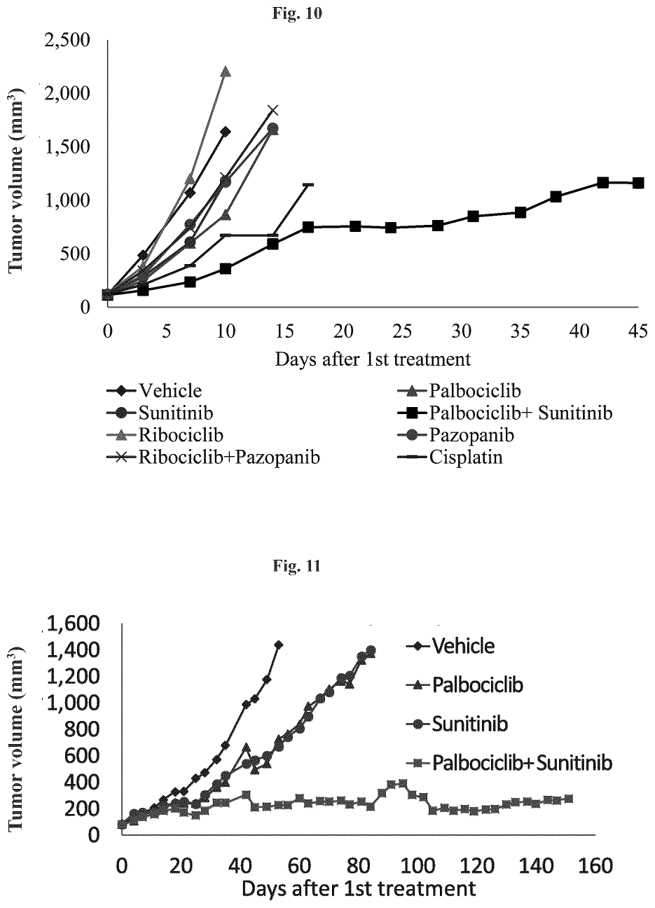

The results are presented in FIG. 10.

As seen in FIG. 10, each one of the drugs, i.e. palbociclib, sunitinib, ribociclib and pazopanib, when applied as monotherapy, had weak to mild effect on tumor size. Combination of ribociclib and pazopanib did not show any additional effect over administration of pazopanib alone. However, the combination of palbociclib and sunitinib showed a synergic effect on TNBC breast cancer. The tumors developed much slower than would have been expect from adding together the effects of monotherapies with each of these drugs alone. Moreover, the combo treatment with palbociclib and sunitinib was even more effective than the well-known treatment with cisplatin. The synergic effect was statistically significant as followed from P-values of Wilcoxon Test (Table 17) and from log rank value of survival test (Table 18).

TABLE 17
Wilcoxon Test P-values

| | | P-value, Wilcoxon |
|---|---|---|
| Treatment | Day | Test (Two-sided) |
| Palbociclib vs combo | 10 | 0.0122 |
| Sunitinib vs combo | 10 | 0.0367 |
| Mean Palbociclib | 10 | 0.0122 |
| Sunitinib vs combo | | |
| Ribociclib vs combo | 10 | 0.1437 |
| Pazopanib vs combo | 10 | 0.6761 |
| Mean Ribociclib | 10 | 0.1437 |
| Pazopanib vs combo | | |

TABLE 18
Log-Rank test

| | Log- |
|---|---|
| Treatment | Rank |
| Palbociclib vs combo | 0.0158 |
| Sunitinib vs combo | 0.0495 |

### Example 3D. Efficacy of the Palbociclib+Sunitinib Combo Treatment of Breast Cancer

Triple negative breast cancer (TNBC) tissue from a different patient was grown in NRG mice, then the tumors were collected, dissociated and implanted in new NRG mice for drug efficacy study. When tumors reached a size of 60-160 mm³, mice were assigned to treatment groups based on the tumor size, tumor growth rate and body weight, creating groups with similar average of these parameters (Table 19). Drugs dosing was initiated at the same day of randomization, denoted as Day 0. The experiment lasted 151 days. Tumor volume was measured (PO: per os).

TABLE 19
Study design

| Group | | | # of treatments | Treatment | # of mice |
|---|---|---|---|---|---|
| # | Tested drugs | | per week | method | per group |
| 1 | Control (vehicle) | | 5 | PO | 5 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 6 |
| | Sunitinib 50 | mg/kg | | | |

The results are presented in FIG. 11.

As seen in FIG. 11, each one of the drugs, i.e., palbociclib and sunitinib administrated as monotherapy had a mild effect on tumor size. The combination of palbociclib and sunitinib showed a synergic effect on TNBC breast cancer. Tumors development was almost completely inhibited for the whole length of the experiment. The synergic effect was statistically significant as followed from P-values of Wilcoxon Test (Table 20).

TABLE 20
Wilcoxon Test P-values

| | | P-value, Wilcoxon |
|---|---|---|
| Treatment | Day | Test (Two-sided) |
| Palbociclib vs combo | 84 | 0.0081 |
| Sunitinib vs combo | 84 | 0.0142 |
| Mean Palbociclib | 84 | 0.0081 |
| Sunitinib vs combo | | |

### Example 3E. Efficacy of the Palbociclib+Sunitinib Combo Treatment of Breast Cancer

Breast cancer tissue from yet another patient was grown in NRG mice, then tumors were collected, dissociated and implanted in new NRG mice for drug efficacy study. When tumors reached a size of 60-105 mm³, mice were assigned to treatment groups based on the tumor size, tumor growth rate and body weight, creating groups with similar averages of these parameters (Table 21). Drugs dosing was initiated at the same day of randomization, denoted as Day 0. The experiment lasted 77 days. Treatment with 5-FU administrated intraperitoneally (IP) served as positive control. Tumor volume was measured (PO: per os; IP: intraperitoneal).

TABLE 21
Study Design

| Group | | | # of treatments | Treatment | # of mice |
|---|---|---|---|---|---|
| # | Tested drugs | | per week | method | per group |
| 1 | Control (vehicle) | | 5 | PO | 6 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 5 |
| | Sunitinib 50 | mg/kg | | | |
| 5 | 5-FU 30 | mg/kg | 2 | IP | 5 |

The results are presented in FIG. 12. As seen in FIG. 12, each one of the drugs, i.e., palbociclib and sunitinib administrated as monotherapies attenuate tumor growth. However, the combination of palbociclib and sunitinib showed a synergic effect on breast cancer. It inhibited tumor growth for more than 40 days. The synergic effect was statistically significant as followed from P-values of Wilcoxon Test (Table 22) and from log rank value of survival test (Table 23). Moreover, the combo treatment was much more effective than 5-FU anti-tumor drug.

TABLE 22
Wilcoxon Test P-values

| | | P-value, Wilcoxon |
|---|---|---|
| Treatment | Day | Test (Two-sided) |
| Palbociclib vs combo | 17 | 0.0122 |
| Sunitinib vs combo | 17 | 0.0122 |
| Mean Palbociclib | 17 | 0.0122 |
| Sunitinib vs combo | | |

TABLE 23
Log-rank values

| | Log- |
|---|---|
| Treatment | Rank |
| Palbociclib vs combo | 0.0486 |
| Sunitinib vs combo | 0.0495 |

A cancer sample from the same patient was used to elucidate the effect of dose on tumor size. The cancer tissue was treated as described above and implanted into mice according to the study indicated in Table 24.

TABLE 24
Study Design

| Group | | | # of treatments | Treatment | # of mice |
|---|---|---|---|---|---|
| # | Tested drugs | | per week | method | per group |
| 1 | Control (vehicle) | | 5 | PO | 6 |
| 2 | Palbociclib 100 | mg/kg + | 5 | PO | 5 |
| | Sunitinib 50 | mg/kg | | | |
| 3 | Palbociclib 50 | mg/kg + | 5 | PO | 5 |
| | Sunitinib 12.5 | mg/kg | | | |
| 4 | Palbociclib 75 | mg/kg + | 5 | PO | 5 |
| | Sunitinib 25 | mg/kg | | | |

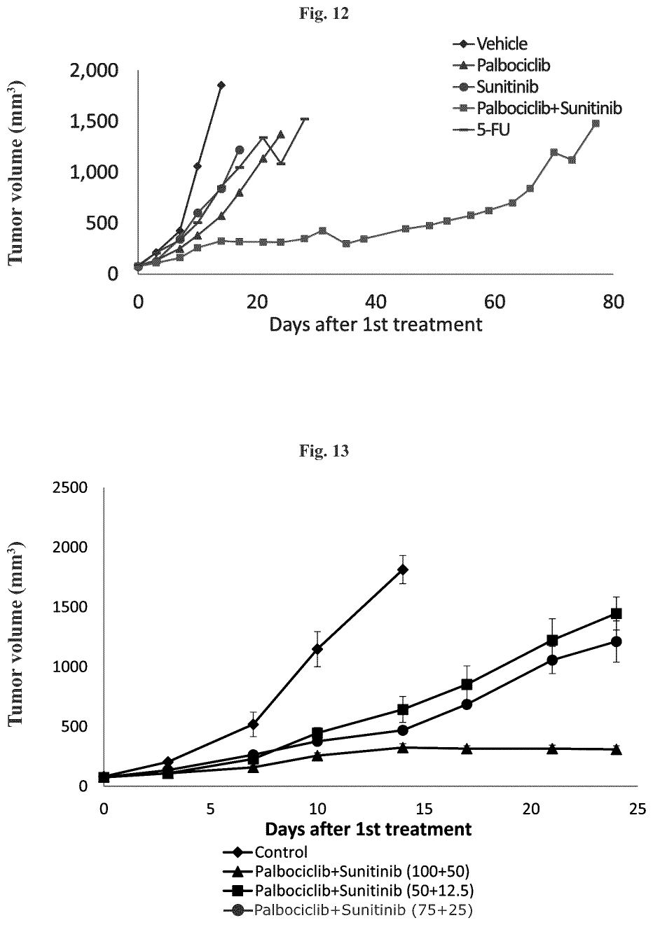

The results are presented in FIG. 13.

As seen in the figure, up to day 15 all doses in the combo treatment provided a significant inhibition of tumor growth compared to control. Tumor volume (mm³) measured on day 15: control=1800, max dose=325, mid dose=470, min. dose=644). On day 24, maximum doses of the combination resulted in continuous inhibition of tumor growth (tumor volume=310 mm³), while in the minimum and mid doses the tumors progressed gradually. Clearly, the combination of palbociclib+sunitinib had a dose response effect on tumor growth inhibition.

### Example 4A. Efficacy of the Palbociclib+Sunitinib and Palbociclib+Sorafenib Combo Treatments of Lung Cancer

Fresh tumor sample of poorly differentiated carcinoma was collected by biopsy from lung and grown in immunocompromised mice. Then, the grown tumor cells were collected, dissociated and implanted subcutaneously into NRG mice necks as described in Material and Methods. When tumors reached a size of 60-160 mm³, mice were assigned to treatment groups based on the tumor size, tumor growth rate and body weight, creating groups with similar averages of these parameters (Table 25). Drugs dosing was initiated at the same day of randomization, denoted as Day 0. The experiment lasted 45 days. Tumor volume was measured (PO: per os).

TABLE 25
Study Design

| Group | | | # of treatments | Treatment | # of mice |
|---|---|---|---|---|---|
| # | Tested drugs | | per week | method | per group |
| 1 | Control (vehicle) | | 5 | PO | 5 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 5 |
| | Sunitinib 50 | mg/kg | | | |
| 5 | Palbociclib 100 | mg/kg + | 5 | PO | 5 |
| | Sorafenib 20 | mg/kg | | | |
| 6 | Sorafenib 20 | mg/kg | 5 | PO | 5 |

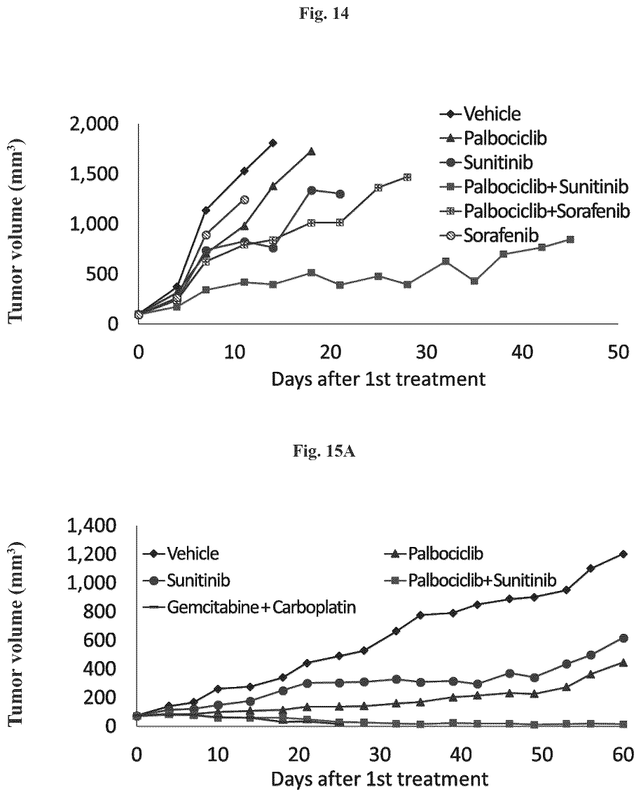

The results are presented in FIG. 14.

As seen in the figure, each one of the drugs, i.e. palbociclib, sunitinib, and sorafenib, administered as monotherapies had weak to mild effect on tumor size. The combination of palbociclib and sorafenib had a week synergic effect which was much more evident after day 14. The combination of palbociclib and sunitinib showed a strong synergic effect on lung cancer. Under this treatment, tumors developed much slower than would have been expected from the mean of monotherapies effects obtained for palbociclib and sunitinib, each alone. The synergic effect of palbociclib and sunitinib combination was statistically significant as followed from P-values of Wilcoxon Test (Table 26).

TABLE 26
Wilcoxon Test P-values

| | | P-value, Wilcoxon |
|---|---|---|
| Treatment | Day | Test (Two-sided) |
| Palbociclib vs combo | 18 | 0.0200 |
| Sunitinib vs combo | 18 | 0.0122 |
| Mean Palbociclib | 18 | 0.0122 |
| Sunitinib vs combo | | |

### Example 4B. Efficacy of the Palbociclib+Sunitinib Combo Treatment of Lung Cancer

Fresh tumor sample of lung adenocarcinoma (KRAS mutation G12V) was collected by biopsy from lung and grown in immunocompromised mice. Then, the grown tumors were collected, dissociated and implanted subcutaneously into NRG mice necks as described in Material and Methods. When tumors reached a size of 60-100 mm³, mice were assigned to treatment groups based on the tumor size, tumor growth rate and body weight (BW), creating groups with similar averages of these parameters (Table 27). Drugs dosing was initiated at the same day of randomization, denoted as Day 0. The experiment lasted 45 days. Tumor volume was measured (PO: per os; IP: intraperitoneal).

TABLE 27
Study Design

| Group | | | # of treatments | Treatment | # of mice |
|---|---|---|---|---|---|
| # | Tested drugs | | per week | method | per group |
| 1 | Control (vehicle) | | 5 | PO | 6 |
| 2 | Palbociclib 100 | mg/kg | 5 | PO | 5 |
| 3 | Sunitinib 50 | mg/kg | 5 | PO | 5 |
| 4 | Palbociclib 100 | mg/kg + | 5 | PO | 6 |
| | Sunitinib 50 | mg/kg | | | |
| 5 | Gemcitabine 25 | mg/kg + | 2 | IP | 5 |
| | Carboplatin 50 | mg/kg | 1 | | |

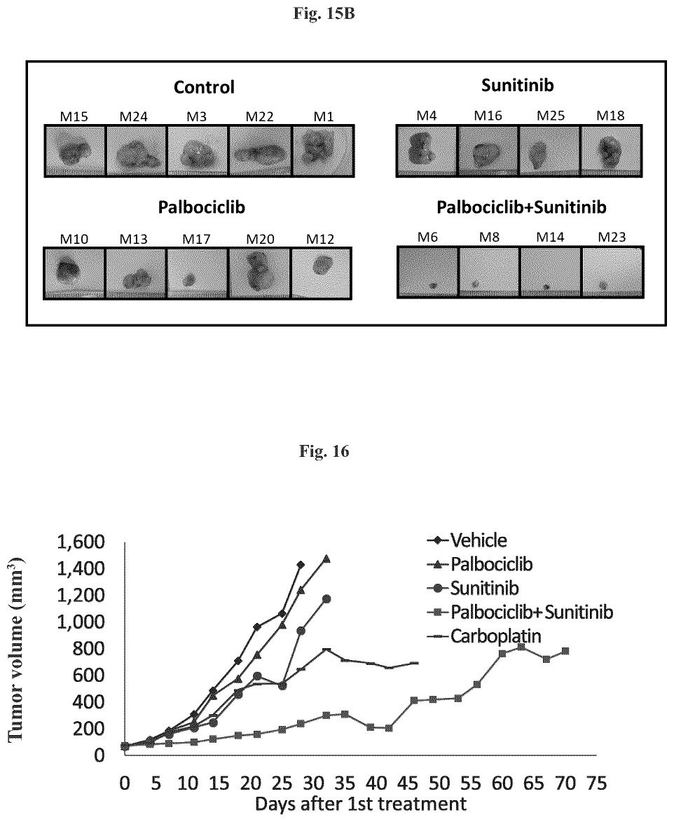

The results are presented in FIGS. 15A-15B.

As seen in FIG. 15A, both palbociclib and sunitinib administered as monotherapies effectively reduced tumor size. Nevertheless, over time (at day 50 onwards) both these monotherapies lost their effects. On the other hand, combination of palbociclib and sorafenib had a much stronger effect in reducing the initial size of the tumor and completely preventing its proliferation. The co-administration of palbociclib and sunitinib had a strong synergic effect on lung cancer. It was as effective as the SOC treatment with gemcitabine+carboplatin. However, the combination of palbociclib and sunitinib was significantly safer for the mice compared to the SOC treatment. On day 25, the SOC group were excluded from the experiment due to toxicity with severe clinical signs (weakness and loss of weight), whereas in the combo group, all 6 mice did not present any toxicity signs until day 60. The synergic effect of palbociclib and sunitinib combination was statistically significant as followed from P-values of Wilcoxon Test (Table 28).

FIG. 15B shows tumors excised from the mice on day 60. It can be clearly seen that the tumors from the combination group were significantly smaller compared to all the other groups.

TABLE 28
Wilcoxon Test P-values

| | | P-value, Wilcoxon |
|---|---|---|
| Treatment | Day | Test (Two-sided) |
| Palbociclib vs combo | 60 | 0.0195 |
| Sunitinib vs combo | 60 | 0.0294 |
| Mean Palbociclib | 60 | 0.0195 |
| Sunitinib vs combo | | |

### Example 5A. Efficacy of the Palbociclib+Sunitinib Combo Treatment of Ovarian Cancer

Fresh tumor sample of metastatic neuroendocrine ovarian cancer was grown in NRG mice, then collected, dissociated and implanted in new NRG mice for drug efficacy study. When tumors reached a size of 60-120 mm³, mice were assigned to treatment groups based on the tumor size, tumor growth rate and body weight, creating groups with similar averages of these parameters (Table 29). Drugs dosing was initiated at the same day of randomization, denoted as Day 0. The experiment lasted 70 days. Tumor volume was measured (PO: per os; IP: intraperitoneal).

TABLE 29
Study Design

| | | # of | | # of |
|---|---|---|---|---|
| Group | | treatments | Treatment | mice per |
| # | Tested drugs | per week | method | group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Carboplatin 50 mg/kg | 2 | IP | 5 |

The results are presented in FIG. 16.

As seen in the figure, the combination of palbociclib and sunitinib had a strong synergic effect on ovarian cancer. Tumor growth was inhibited and stabilized until ˜day 42 by the combo treatment, after which the tumors started to develop resistance to therapy. Nevertheless, the tumors developed much slower than would have been expected from combining the monotherapies effects of each of palbociclib and sunitinib. In fact, the combo treatment was even more efficient than the well-known treatment with carboplatin. The synergic effect of palbociclib and sunitinib combo treatment was statistically significant as followed from P-values of Wilcoxon Test (Table 30). In addition, in the carboplatin treatment group, 3 out of 5 tumors formed ulcers between day 28 to 46, which led to termination of the study for this group at day 46, while in the combination group at day 46 only one mouse (out of 5) was excluded due to ulcer. Thus, the combo treatment not only provided synergic treatment of the cancer but was much safer than the SOC treatment.

TABLE 30
Wilcoxon Test P-values

| | | P-value, Wilcoxon |
|---|---|---|
| Treatment | Day | Test (Two-sided) |
| Palbociclib Vs combo | 32 | 0.0122 |
| Sunitinib Vs combo | 32 | 0.0369 |
| Mean Palbociclib | 32 | 0.0122 |
| Sunitinib Vs combo | | |

### Example 5B. Efficacy of the Palbociclib+Sunitinib and Palbociclib+Sorafenib Combo Treatments of Ovarian Cancer

Fresh tumor sample of metastatic high grade serous carcinoma with BRCA1 mutation (ovarian cancer) was grown in NRG mice, then collected, dissociated and implanted in new NRG mice for drug efficacy study. When tumors reached a size of 60-150 mm³, mice were assigned to treatment groups based on the tumor size, tumor growth rate and body weight, creating groups with similar averages of these parameters (Table 31). Drugs dosing was initiated at the same day of randomization, denoted as Day 0. The experiment lasted 172 days. Tumor volume was measured. On day 172, 3 mice in the combo group (palbociclib+sunitinib) were still alive: 2 mice showed stable diseases, and in one mouse the tumor developed resistance and started to grow. In all groups, there were 2-3 mice that were removed from study due to ulcer or weakness, starting from day 38 (PO: per os).

TABLE 31
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg | 5 | PO | 6 |
| 3 | Sunitinib 50 mg/kg | 5 | PO | 6 |
| 4 | Palbociclib 100 mg/kg + | 5 | PO | 6 |
| | Sunitinib 50 mg/kg | | | |
| 5 | Palbociclib 100 mg/kg + | 5 | PO | 5 |
| | Sorafenib 20 mg/kg | | | |
| 6 | Sorafenib 20 mg/kg | 5 | PO | 4 |

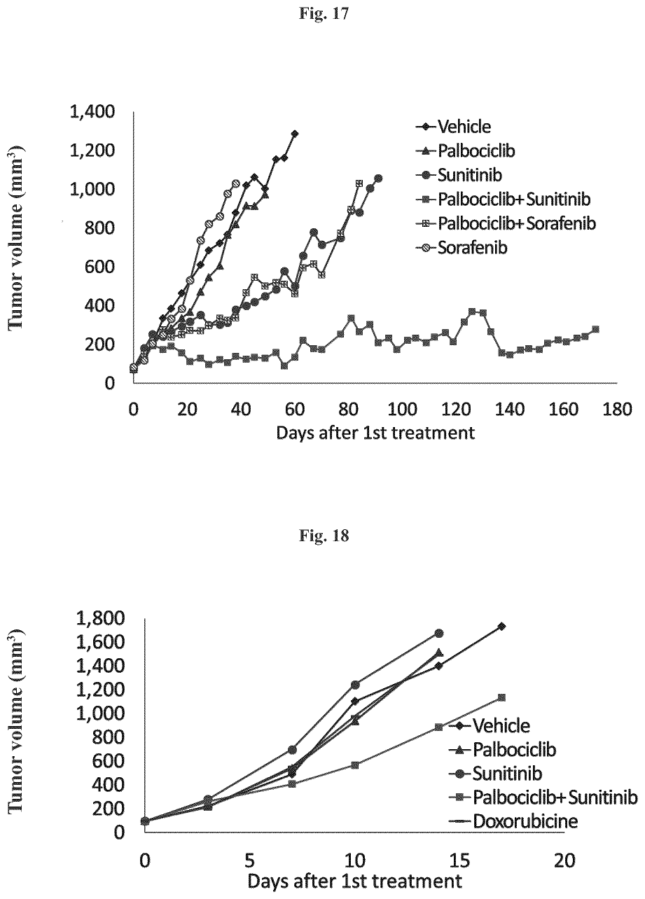

The results are presented in FIG. 17. As seen in the figure, treatment with palbociclib or sorafenib as monotherapies had no effect on tumor growth, whereas sunitinib attenuated significantly tumor growth. It is further seen that both combinations: palbociclib with sunitinib and palbociclib with sorafenib, had a significant synergistic effect on ovarian tumor growth. Treatment with a combination of palbociclib and sunitinib was much more efficient than with a combination of palbociclib and sorafenib. The synergic effect of palbociclib and sunitinib combination treatment was statistically significant as followed from P-values of Wilcoxon Test (Table 32).

TABLE 32
Wilcoxon Test P-values

| | | P-value, Wilcoxon |
|---|---|---|
| Treatment | Day | Test (Two-sided) |
| Palbociclib Vs combo | 49 | 0.0200 |
| Sunitinib Vs combo | 49 | 0.0137 |
| Mean Palbociclib | 49 | 0.0081 |
| Sunitinib Vs combo | | |
| Sunitinib Vs combo | 91 | 0.0369 |

### Example 6. Efficacy of the Palbociclib+Sunitinib Combo Treatment of Carcinosarcoma

Fresh tumor sample of carcinosarcoma was grown in NRG mice, then tumors were collected, dissociated and implanted in new NRG mice for drug efficacy study. When tumors reached a size of 60-140 mm³, mice were assigned to treatment groups based on the tumor size, tumor growth rate and body weight, creating groups with similar averages of these parameters (Table 33). Drugs dosing was initiated at the same day of randomization, denoted as Day 0. The experiment lasted 17 days. Tumor volume was measured (PO: per os; IP: intraperitoneal).

TABLE 33
Study Design

| | | # of | | # of |
|---|---|---|---|---|
| Group | | treatments | Treatment | mice per |
| # | Tested drugs | per week | method | group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Doxorubicine 4 mg/kg | 1 | IP | 4 |

The results are presented in FIG. 18.

As seen in the figure, none of the tested drugs when administered alone had any effect on tumor growth. However, in the combination group several mice responded very strongly to the treatment. It is thus evident that a combination of palbociclib with sunitinib inhibited tumor growth and provided profound synergistic inhibitory effect on carcinosarcoma. The synergistic effect can be clearly seen from the graphs. Furthermore, the combo treatment was much more effective than treatment with the known anticancer drug doxorubicin.

### Example 7. Efficacy of the Palbociclib+Sunitinib Combo Treatment of Pancreatic Cancer

Fresh adenocarcinoma tumor (pancreatic cancer) was grown in NRG mice, then tumors were collected, dissociated and implanted in new NRG mice for drug efficacy study. When tumors reached a size of 70-170 mm³, mice were assigned to treatment groups based on the tumor size, tumor growth rate and body weight, creating groups with similar averages of these parameters (Table 34). Drugs dosing was initiated at the same day of randomization, denoted as Day 0. The experiment lasted 66 days. Tumor volume was measured (PO: per os; IP: intraperitoneal).

TABLE 34
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg | 5 | PO | 6 |
| 3 | Sunitinib 50 mg/kg | 5 | PO | 6 |
| 4 | Palbociclib 100 mg/kg + | 5 | PO | 6 |
| | Sunitinib 50 mg/kg | | | |
| 5 | Gemcitabine 25 mg/kg + | 2 | IP | 6 |
| | Cisplatin 3 mg/kg | | | |

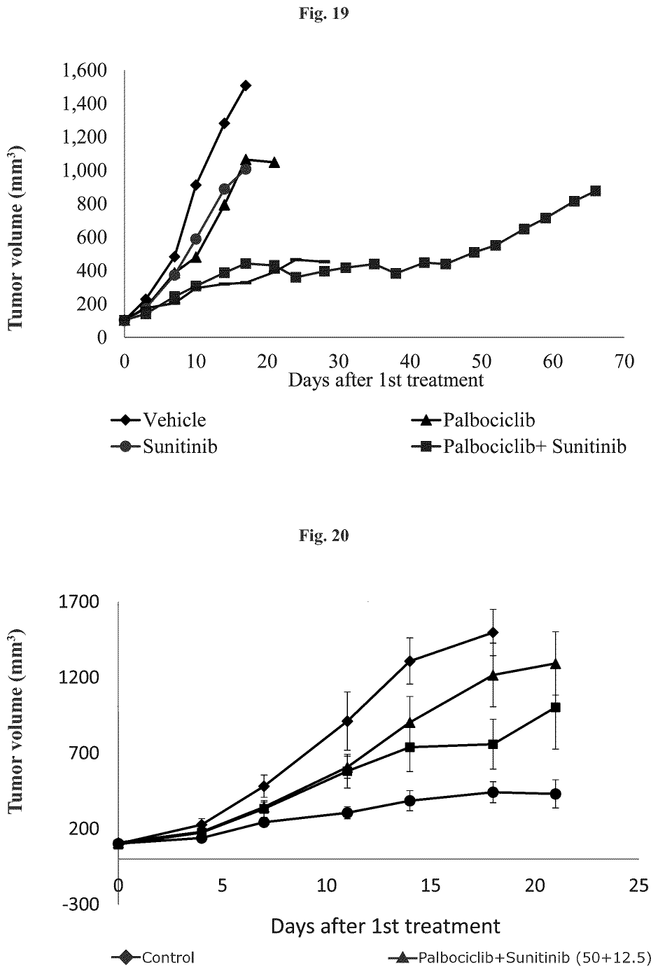

The results are presented in FIG. 19.

As seen in the figure, palbociclib and sunitinib when administered alone (as monotherapies) had moderate effect on the tumor size. However, combination of palbociclib with sunitinib significantly inhibited tumor growth for a prolonged period. Furthermore, the combo treatment had similar efficacy as a combination of two acknowledged anticancer drugs: gemcitabine and cisplatin (SOC treatment). Moreover, the combination of palbociclib and sunitinib was significantly safer to the mice compared to the SOC treatment: on day 28 all mice in the SOC group were excluded from the experiment due to toxicity with severe clinical signs (weakness and loss of weight), whereas in the combination group 4 out of 6 mice were treated without any toxicity signs until day 66. The synergic effect of palbociclib and sunitinib combination was statistically significant as followed from P-values of Wilcoxon Test (Table 35).

TABLE 35
Wilcoxon Test P-values

| | | P-value, Wilcoxon |
|---|---|---|
| Treatment | Day | Test (Two-sided) |
| Palbociclib Vs combo | 17 | 0.0131 |
| Sunitinib Vs combo | 17 | 0.0142 |
| Mean Palbociclib | 17 | 0.0082 |
| Sunitinib Vs combo | | |

The effect of combo treatment was dose dependent as has been further shown. Drug efficacy studies using NRG mice implanted with pancreatic cancer cells were carried out as described above and summarized in Table 36. The experiment lasted 21 days.

TABLE 36
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg + | 5 | PO | 6 |
| | Sunitinib 50 mg/kg | | | |
| 3 | Palbociclib 50 mg/kg + | 5 | PO | 6 |
| | Sunitinib 12.5 mg/kg | | | |
| 4 | Palbociclib 75 mg/kg + | 5 | PO | 6 |
| | Sunitinib 12.5 mg/kg | | | |

The results presented in FIG. 20 show clear dose dependence of the combo treatment. The highest tested dose almost completely inhibited tumor growth.

### Example 8. Efficacy of the Palbociclib+Sunitinib Combo Treatment of Cholangiocarcinoma

Fresh tumor sample of cholangiocarcinoma was grown in NRG mice, then tumors were collected, dissociated and implanted in new NRG mice for drug efficacy study. When tumors reached a size of 60-110 mm³, mice were assigned to treatment groups based on the tumor size, tumor growth rate and body weight, creating groups with similar averages of these parameters (Table 37). Drugs dosing was initiated at the same day of randomization, denoted as Day 0. The experiment lasted 87 days. Tumor volume was measured (PO: per os; IP: intraperitoneal).

TABLE 37
Study Design

| | | # of | | # of |
|---|---|---|---|---|
| Group | | treatments | Treatment | mice per |
| # | Tested drugs | per week | method | group |
| 1 | Control (vehicle) | 5 | PO | 5 |
| 2 | Palbociclib 100 mg/kg | 5 | PO | 5 |
| 3 | Sunitinib 50 mg/kg | 5 | PO | 5 |
| 4 | Palbociclib 100 mg/kg + | 5 | PO | 5 |
| | Sunitinib 50 mg/kg | | | |
| 5 | Gemcitabine 25 mg/kg + | 2 | IP | 4 |
| | Cisplatin 1.5 mg/kg | | | |

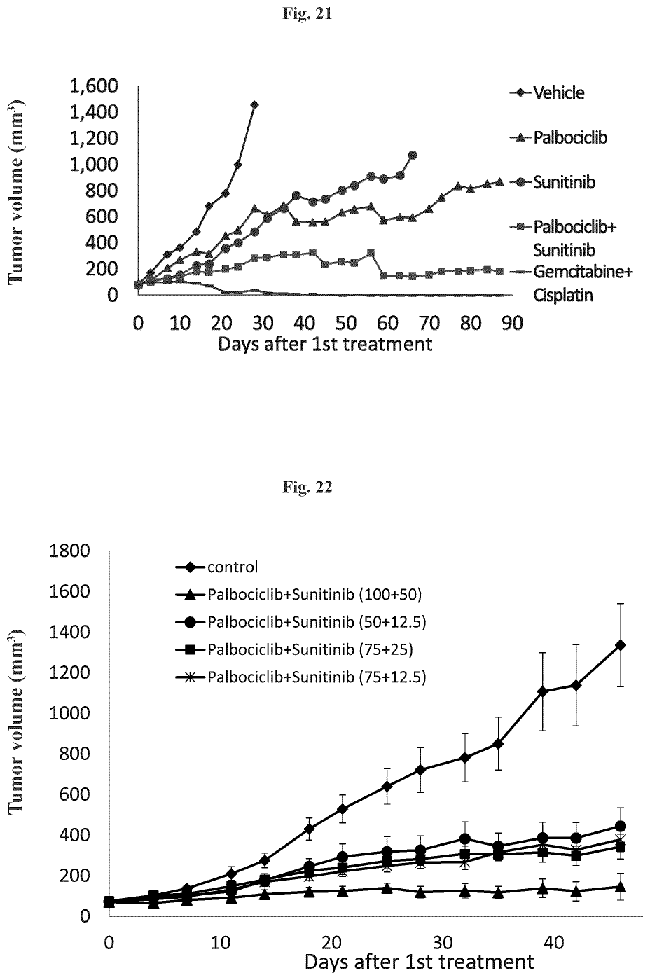

The results are presented in FIG. 21.

As seen in the figure, treatment with each one of palbociclib and sunitinib administered as monotherapy provided similar moderate inhibitory effect on tumor growth. However, the combination of palbociclib with sunitinib synergically inhibited tumor growth for the whole period of the experiment. In fact, treatment with a combination of palbociclib and sunitinib provided similar results to those obtained from the combination of two known anticancer drugs: gemcitabine and cisplatin. The synergic effect of palbociclib and sunitinib combination was statistically significant as followed from P-values of Wilcoxon Test (Table 38).

TABLE 38
Wilcoxon Test P-values

| | | P-value, Wilcoxon |
|---|---|---|
| Treatment | Day | Test (Two-sided) |
| Palbociclib Vs combo | 66 | 0.0200 |
| Sunitinib Vs combo | 66 | 0.0373 |
| Mean Palbociclib | 66 | 0.0200 |
| Sunitinib Vs combo | | |

Dose response was tested for 46 days. The mice were allocated according to Table 39.

TABLE 39
Study Design

| | | # of | | # of |
|---|---|---|---|---|
| Group | | treatments | Treatment | mice per |
| # | Tested drugs | per week | method | group |
| 1 | Control (vehicle) | 5 | PO | 6 |
| 2 | Palbociclib 100 mg/kg + | 5 | PO | 7 |
| | Sunitinib 50 mg/kg | | | |
| 3 | Palbociclib 50 mg/kg + | 5 | PO | 7 |
| | Sunitinib 12.5 mg/kg | | | |
| 4 | Palbociclib 75 mg/kg + | 5 | PO | 6 |
| | Sunitinib 25 mg/kg | | | |
| 5 | Palbociclib 75 mg/kg + | 5 | PO | 6 |
| | Sunitinib 12.5 mg/kg | | | |

The results presented in FIG. 22 clearly show dose dependence of the combo treatment. All tested combinations were highly effective, while the highest dose regiment provided the most profound effect in inhibiting tumor growth.

### Example 9. Efficacy of the Palbociclib+Sunitinib Combo Treatment and Sorafenib Monotherapy in Hepatocellular Carcinoma Model

Fresh tumor sample of hepatitis C virus (HCV) positive hepatocellular carcinoma was grown in NRG mice, then tumors were collected, dissociated and implanted in new NRG mice for drug efficacy study. When tumors reached a size of 60-260 mm³, mice were assigned to treatment groups based on the tumor size, tumor growth rate and body weight, creating groups with similar averages of these parameters (Table 40). Drugs dosing was initiated at the same day of randomization, denoted as Day 0. The experiment lasted 14 days. Tumor volume was measured (PO: per os).

TABLE 40
Study Design

| | | # of | | # of |
|---|---|---|---|---|
| Group | | treatments | Treatment | mice per |
| # | Tested drugs | per week | method | group |
| 1 | Control (vehicle) | 5 | PO | 7 |
| 2 | Palbociclib 100 mg/kg | 5 | PO | 7 |
| 3 | Sunitinib 50 mg/kg | 5 | PO | 8 |
| 4 | Palbociclib 100 mg/kg + | 5 | PO | 7 |
| | Sunitinib 50 mg/kg | | | |
| 5 | Sorafenib 20mg/kg | 5 | PO | 6 |

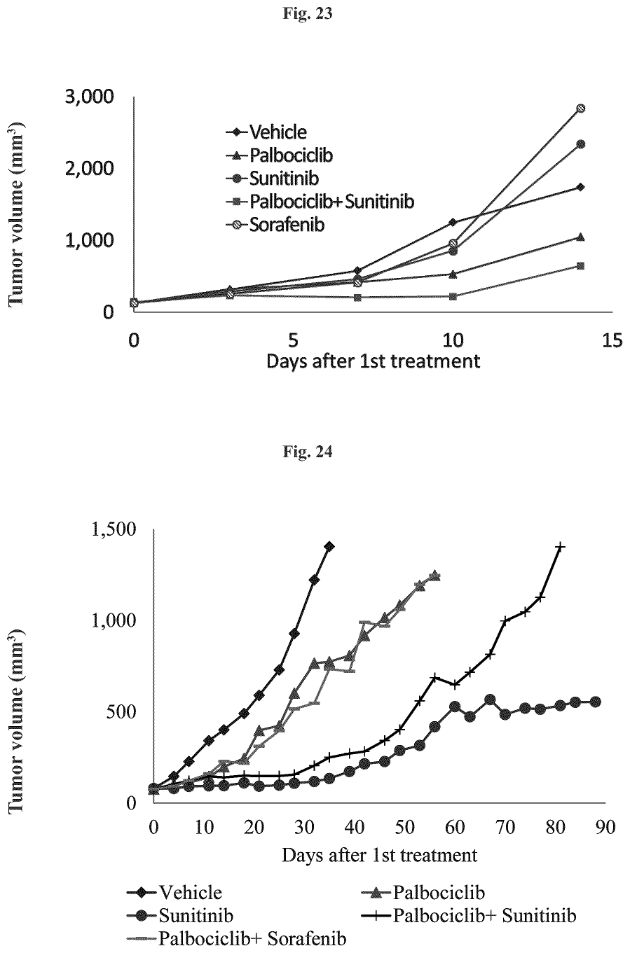

The results are presented in FIG. 23.

As seen in the figure, treatment with a combination of palbociclib and sunitinib was only slightly better than that obtained with palbociclib alone. No synergic effect was seen for the combination as evident from the high P-values of Wilcoxon Test (Table 41).

TABLE 41
Wilcoxon Test P-values

| | | P-value, Wilcoxon |
|---|---|---|
| Treatment | Day | Test (Two-sided) |
| Palbociclib Vs combo | 14 | 0.6171 |
| Sunitinib Vs combo | 14 | 0.0306 |
| Mean Palbociclib | 14 | 0.0169 |
| Sunitinib Vs combo | | |

### Example 10. Efficacy of the Palbociclib+Sunitinib, Palbociclib+Sorafenib, and Ribociclib+Pazopanib Combo Treatments of Appendix Carcinoma

Fresh tumor sample of appendix carcinoma was grown in NRG mice, then tumors were collected, dissociated and implanted in new NRG mice for drug efficacy study. When tumors reached a size of 60-140 mm³, mice were assigned to treatment groups based on the tumor size, tumor growth rate and body weight, creating groups with similar averages of these parameters (Table 42). Drugs dosing was initiated at the same day of randomization, denoted as Day 0. The experiment lasted 35 days. Tumor volume was measured (PO: per os).

TABLE 42
Study Design

| | | # of | | # of |
|---|---|---|---|---|
| Group | | treatments | Treatment | mice per |
| # | Tested drugs | per week | method | group |
| 1 | Control (vehicle) | 5 | PO | 10 |
| 2 | Palbociclib 100 mg/kg | 5 | PO | 6 |
| 3 | Sunitinib 50 mg/kg | 5 | PO | 5 |
| 4 | Palbociclib 100 mg/kg + | 5 | PO | 6 |
| | Sunitinib 50 mg/kg | | | |
| 5 | Palbociclib 100 mg/kg + | 5 | PO | 5 |
| | Sorafenib 20 mg/kg | | | |
| 6 | Ribociclib 100 mg/kg | 5 | PO | 6 |
| 7 | Pazopanib | 5 | PO | 6 |
| 8 | Ribociclib 100 mg/kg + | 5 | PO | 6 |
| | Pazopanib 100 mg/kg | | | |

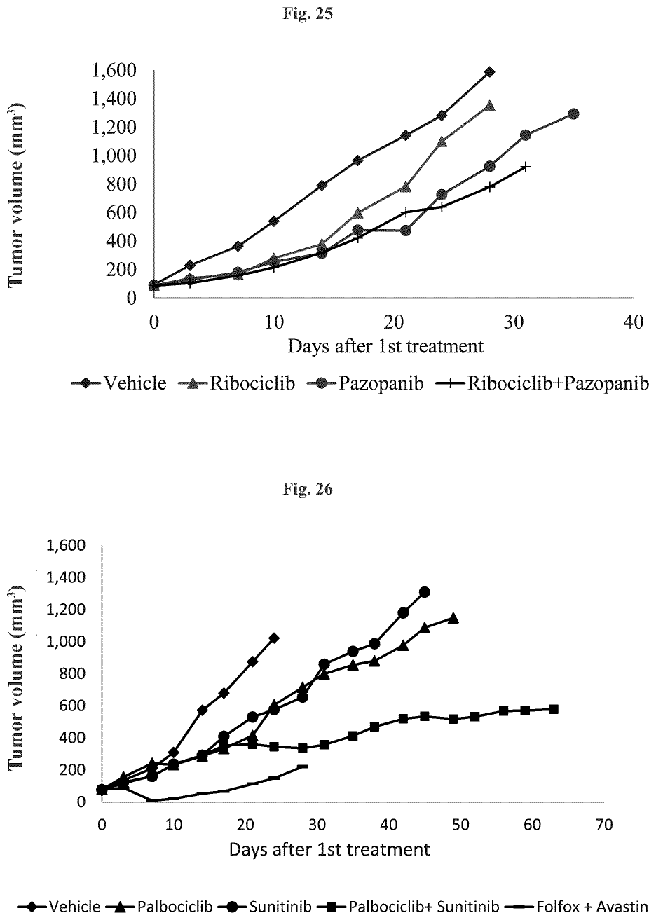

The results are presented in FIGS. 24 and 25.

As seen in these figures, none of the combinations, i.e., palbociclib and sunitinib, palbociclib and sorafenib, or ribociclib and pazopanib, had a synergic effect. In fact, sunitinib alone worked better than its combination with palbociclib. Apparently, sorafenib in combination with palbociclib had no effect as the combo behaved exactly as palbociclib alone. Combination of ribociclib and pazopanib behaved as pazopanib alone. Therefore, in this type of cancer, the combo treatment had no added value over monotherapies.

### Example 11. Efficacy of the Palbociclib+Sunitinib Combo Treatment of Ewing Sarcoma

Fresh tumor sample of Ewing Sarcoma was grown in NRG mice, then tumors were collected, dissociated and implanted in new NRG mice for drug efficacy study. When tumors reached a size of 60-100 mm³, mice were assigned to treatment groups based on the tumor size, tumor growth rate and body weight, creating groups with similar averages of these parameters (Table 43). Drugs dosing was initiated at the same day of randomization, denoted as Day 0. The experiment lasted 35 days. Tumor volume was measured (PO: per os; IP: intraperitoneal).

TABLE 43
Study Design

| | | # of | | # of |
|---|---|---|---|---|
| Group | | treatments | Treatment | mice per |
| # | Tested drugs | per week | method | group |
| 1 | control (vehicle) | 5 | PO | 5 |
| 2 | palbociclib 100 mg/kg | 5 | PO | 5 |
| 3 | sunitinib 50 mg/kg | 5 | PO | 5 |
| 4 | palbociclib 100 mg/kg + | 5 | PO | 5 |
| | sunitinib 50 mg/kg | | | |
| 5 | ifosfamid 60 mg/kg+ | 2 | IP | 6 |
| | doxorubicine 4 mg/kg | 1 | | |

The results are presented in FIG. 26. As seen in the figure, palbociclib and sunitinib, when administered alone, had moderate effect on the tumor size. However, a combination of palbociclib with sunitinib significantly inhibited tumor growth for a prolonged period. The combo treatment had lower efficacy than the treatment with folfox and avastin (SOC). Nevertheless, with time, the combo treatment's efficiency was equivalent to the SOC. Most importantly, the combo treatment was much less toxic than the SOC. Mice of SOC treatment were very weak and lost weight, and were excluded from the study from day 7 to day 42. On the other hand, all mice of the combo treatment reached the end of the experiment.

### Example 11. Clinical Trials: Treatment of Adenoid Cystic Carcinoma (ACC) Patients With a Combination of Palbociclib and Sunitinib

Three patients diagnosed with adenocystic carcinoma (herein interchangeable with adenoid cystic carcinoma (ACC)) enrolled in a clinical trial whereby they were treated with a combination of palbociclib and sunitinib. The treatment delivered to all patients was oral: palbociclib 75 mg per day for 5 consecutive days, every seven days; sunitinib 25 mg per day for the first 5 days and then escalated to 37.5 mg every day for 5 days, repeated every 7 days. Dose modifications were done based upon toxicity.

### Patient 1

A 55-year-old male married and father of 3.

In March 2023, the patient was diagnosed with a base of tongue adenocystic carcinoma, locally advanced and inoperable. He was treated with proton radiotherapy and carbon beam ion therapy to the base of tongue (carbon ion irradiation, whereby charged carbon nuclei are directed at the tumor). Within 4 months following this treatment, the patient developed metastatic disease that spread to the lung and bone. Also, the patient developed soft tissue edema post radiotherapy.

In March 2024, the patient started palbociclib and sunitinib combo therapy. Within 14 days of treatment, the patient showed clinical improvement in the edema, and increased appetite and energy. Pet CT post 2 months of disease revealed stability of the disease. This patient is still under treatment.

### Patient 2

A 47-year-old female married and mother of 2.

In November 2020, diagnosed with high-grade malignancy in the left upper jaw area, a tumor thought to arise from the maxillary sinus, i.e., the upper jawbone, which also forms part of the orbit (eye socket) and nasal cavity (the sinuses are air-filled spaces within the bones around the nose, and the nasal cavity is the space inside the nose). The cancer was primarily classified as an adenoid cystic carcinoma but exhibited squamous cell characteristics (namely, the tumor had features of both adenoid cystic carcinoma and squamous cell carcinoma), making it more aggressive than typical ACC or high grade ACC. Squamous differentiation means that some of the cancer cells have developed characteristics similar to squamous cells (flat cells that are typically found in the outer layer of the skin and lining of certain organs, including the respiratory and digestive tracts). The presence of squamous differentiation often indicates that the tumor cells are more diverse in type, and this variation can influence the behavior and treatment response of the tumor. This tumor was challenging to treat due to its complex cellular makeup.

In March 2021, the patient underwent extensive surgery, including removal of positive margins. Aggressive progression of disease post-surgery was observed both locoregional and systemic. Treatments with cisplatin, taxotere and pembrolizumab, followed by oxaliplatin and 5FU, failed. The patient developed severe trismus with almost no possibility for feeding.

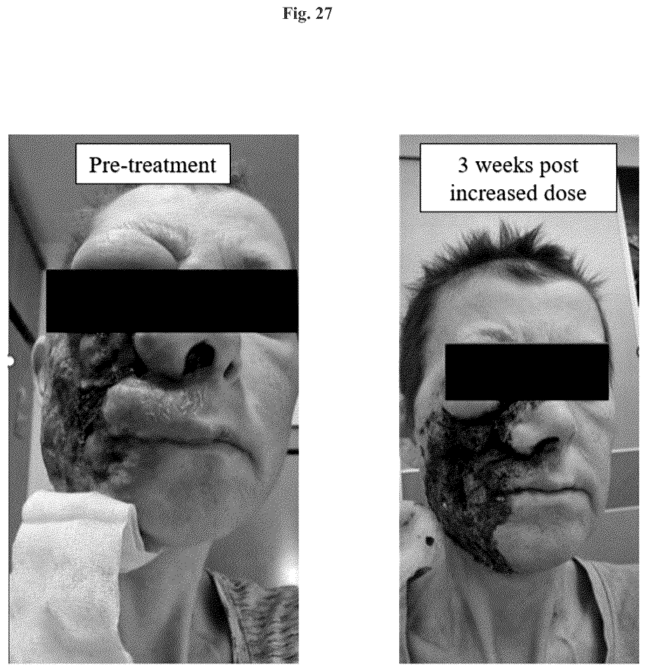

The patient started combo therapy protocol comprising palbociclib and sunitinib, and within 10 days showed marked reduction in tumor size and reduction of the pain. Lamentable, the patients died 3 weeks on study due to aspiration pneumonia. Photographs of the patient before combo treatment and 3 weeks post combo treatment with increased dose are shown in FIG. 27.

### Patient 3

A 59-year-old female.

In 2013, diagnosed with adenoid cystic carcinoma T3N0M0, a case where the tumor is relatively large or has started to invade nearby structures (category T3, namely, the tumor is greater than 4 cm in size or has extended into the surrounding anatomical structures. However, it has not invaded critically nearby structures like the base of the skull), there is no lymph node involvement (N0), and there are no signs of distant metastasis (M0). This staging implies an advanced local tumor but without regional or distant spread, which could impact the treatment plan and prognosis.

The patient underwent surgery followed by radiotherapy. In August 2022, post radiation malignancy was diagnosed. In March 2023, the patient was operated on, but macroscopic (visible) cancer remained after surgery (R2 resection-adenoid cystic carcinoma), necessitating additional treatment. In July 2023, progressive disease was diagnosed, manifested by severe pain in the face and back.

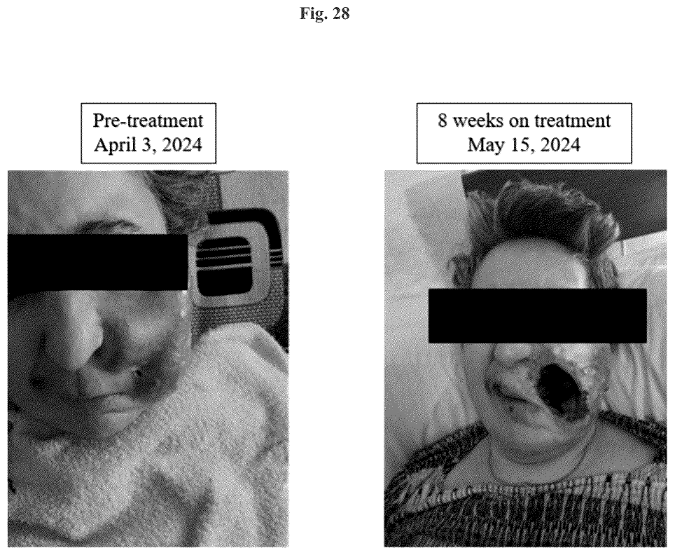

In April 2024, the patient was subjected to combo therapy comprising palbociclib and sunitinib. Within 1 week, all the back pain and face pain disappeared. Within 4 weeks, all the disease disappeared from the large face mass and became a large cavity. Photographs of the patient before, and 8 weeks after combo treatment are shown in FIG. 28.

1. A method for treating adenoid cystic carcinoma (ACC) in a subject in need thereof, comprising administering to the subject a combination of at least one inhibitor of cyclin-dependent kinase 4/6 (CDK 4/6) and at least one multi-targeted receptor tyrosine kinase inhibitor (mtRTKI).

2. The method of claim 1, wherein the at least one CDK 4/6 inhibitor is selected from the group consisting of palbociclib, abemaciclib and ribociclib, and the at least one mtRTKI is selected from the group consisting of sunitinib, sorafenib and pazopanib.

3. The method of claim 2, wherein at least one CDK 4/6 inhibitor is palbociclib.

4. The method of claim 2, wherein at least one mtRTKI is sunitinib.

5. The method of claim 1, wherein the combination comprises palbociclib and sunitinib.

6. The method of claim 5, wherein palbociclib is administered in a dose of from 20 to 250 mg/day.

7. The method of claim 5, wherein sunitinib is administered in a dose of from 10 to 125 mg/day.

8. The method of claim 5, wherein palbociclib is administered in a dose of from 60 to 80 mg/day, and sunitinib is administered in a dose of from 20 to 40 mg/day.

9. The method of claim 1, wherein the combination provides a synergistic effect.

10. The method of claim 1, wherein the at least one CDK 4/6 inhibitor and the at least one mtRTKI are administered simultaneously.

11. The method of claim 1, wherein the at least one CDK 4/6 inhibitor and the at least one mtRTKI are administered sequentially.

12. The method of claim 5, wherein at least one of palbociclib and sunitinib is administered orally.

13. The method of claim 12, wherein each of palbociclib and sunitinib is administered for 5 consecutive days, every seven days.
